(19) United States
(12) Patent Application Publication
(10) Pub. No.: US 2018/0346566 A1
(43) Pub. Date: Dec. 6, 2018

(54) METHODS AND COMPOSITIONS RELATING TO ANTI-TMIGD1/IGPR-2

(71) Applicant: TRUSTEES OF BOSTON UNIVERSITY, Boston, MA (US)

(72) Inventor: Nader RAHIMI, Westwood, MA (US)

(73) Assignee: TRUSTEES OF BOSTON UNIVERSITY, Boston, MA (US)

(21) Appl. No.: 15/572,263

(22) PCT Filed: May 19, 2016
(86) PCT No.: PCT/US16/33209

§ 371 Date: Nov. 7, 2017

Related U.S. Application Data
(60) Provisional application No. 62/163,746, filed on May 19, 2015.

(51) Int. Cl. C07K 16/28; C07K 14/705; A61P 35/00; A61P 13/12

(52) U.S. Cl. CPC C07K 16/2803; C07K 14/70503; A61K 2039/505; A61P 13/12; C07K 2317/75; A61P 35/00

(57) ABSTRACT

The technology described herein is directed to the diagnosis, prognosis, and treatment of conditions of the kidney, e.g., kidney cancer and/or kidney disease, and also relates to methods, compositions and kits comprising an agonist of the TMIGD1 (transmembrane and immunoglobulin containing 1) polypeptide.

### CROSS-REFERENCE TO RELATED APPLICATIONS

This application claims benefit under 35 U.S.C. § 119(e) of U.S. Provisional Application No. 62/163,746 filed May 19, 2015, the contents of which is incorporated herein by reference in its entirety.

### GOVERNMENT SUPPORT

This invention was made with Government Support under Grant No. R01EY017955 awarded by the National Institutes of Health. The U.S. government has certain rights in the invention.

### TECHNICAL FIELD

The technology described herein relates to the diagnosis, prognosis, and treatment of kidney diseases, e.g., cancer, acute injury, or chronic kidney diseases.

### BACKGROUND

Renal cell carcinoma (RCC), the most common urological cancer, is composed of a heterogeneous group of kidney epithelial tumors with variable genetic and clinical outcomes. RCC is a high-risk metastasizing tumor that has a poor prognosis as there are very few tumor markers for renal cancer and it is generally insensitive to conventional chemo and radiotherapies or targeted therapeutics, including anti-angiogenesis drugs and mTOR inhibitors. Effective diagnosis and treatment of RCC will require a better understanding of the underlying pathologies and identification of effective therapeutic targets.

### SUMMARY

As described herein, the inventors have discovered the TMIGD1 (transmembrane and immunoglobulin containing 1) polypeptide plays a key role in the pathology of kidney cancers, such as e.g., RCC, as well as protecting kidney cells from various types of damage, including oxidative damage. Activating TMIGD1 is demonstrated herein to be an effective way of treating both cancer and kidney diseases, and accordingly, an agonists of TMIGD1 that can be used in such therapeutic approaches, methods, compositions and kits are provided.

Aspects of the technology disclosed herein relate to a method of treating kidney disease or kidney cancer in a subject in need thereof, the method comprising administering a TMIGD1 agonist to the subject, for example, where the agonist is selected from the group consisting of: an antibody reagent (e.g., activating anti-TMIGD1 antibody or antigen-binding fragment thereof); a TMIGD1 polypeptide; and a nucleic acid encoding a TMIGD1 polypeptide.

In alternative embodiments, a TMIGD1 agonist is a polypeptide comprising the sequence of SEQ ID NO: 9 (CYR61) or a polypeptide having at least 80% sequence identity to SEQ ID NO: 9.

In some embodiments, the kidney cancer is renal cell carcinoma (RCC), and in some embodiments, the kidney disease is selected from the group consisting of: acute kidney injury (AKI); chronic kidney disease (CKD), kidney cysts, Alport Syndrome, Diabetic Nephropathy, Fabry Disease, Focal Segmental Glomerulosclerosis, Glomerulonephritis, IgA Nephropathy (Berger's Disease), Kidney Stones and Polycystic Kidney Disease (PKD).

As disclosed herein, any antibody reagent can be used, e.g., an activating anti-TMIGD1 antibody or antigen binding fragment thereof. In some embodiments, an activating anti-TMIGD1 antibody is selected from any or a combination of the group consisting of: polyclonal antibody, monoclonal antibody, humanized or chimeric antibody or human monoclonal antibody.

In some embodiments, an activating anti-TMIGD1 antibody or antibody reagent specifically binds to the extracellular domain of the TMIGD1 polypeptide corresponding to amino acids 41-215 of SEQ ID NO: 10. In some embodiments, an activating anti-TMIGD1 antibody or antibody reagent specifically binds to any region of: Ig I (corresponding to amino acids 44-123 of SEQ ID NO: 10), Ig2 (corresponding to amino acids 124-215 of SEQ ID NO: 10) or Ig1 and Ig2. In alternative embodiments, an activating anti-TMIGD1 antibody or antibody reagent blocks the interaction or binding of CYR61 polypeptide with the extracellular domain of the TMIGD1 polypeptide corresponding to amino acids 41-215 of SEQ ID NO: 10.

Another aspect of the technology described herein relates to an isolated activating antibody which specifically binds the CYR61-binding domain of TMIGD1, thereby activating TMIGD1, where the CYR61 binding domain is located in the extracellular domain of the TMIGD1 polypeptide corresponding to amino acids 29-215 of SEQ ID NO: 10, or located in the extracellular domain of the TMIGD1 comprising the Ig1 and/or Ig2 domains corresponding to amino acids 41-215 of SEQ ID NO: 10. In some embodiments, an activating antibody which specifically binds the CYR61-binding domain of TMIGD1specifically binds to at least 1 or more amino acids in any region of: Ig1 (corresponding to amino acids 44-123 of SEQ ID NO: 10), Ig2 (corresponding to amino acids 124-215 of SEQ fD NO: 10) or Ig1 and Ig2. In some embodiments, an activating antibody which specifically binds the CYR61-binding domain of TMIGD1 competes with CYR61 for specific binding to TMIGD1 (i.e., such an activating antibody which specifically binds the CYR61 -binding domain of TMIGD1 competitively inhibits CYR61 bind to the extracellular domain of TMIGD1).

In all aspects of the technology described herein, an activating anti-TMIGD1 antibody or antigen binding fragment thereof, or an activating antibody which specifically binds the CYR61-binding domain of TMIGD1 is selected from: a polyclonal antibody, a monoclonal antibody, a human antibody, a humanized antibody a chimeric antibody or antigen-binding fragments thereof.

Another aspect of the technology described herein relates to a TMIGD1 agonist comprising a nucleic acid sequence encoding a TMIGD1 polypeptide, for example, a TMIGD1 polypeptide having the amino acid sequence of SEQ NO: 10 or 11, or having a the sequence of at least 80% sequence identity to SEQ ID NO: 10 or 11. In some embodiments, a TMIGD1 agonist comprises a nucleic acid sequence encoding a TMIGD1 polypeptide, for example, a TMIGD1 polypeptide comprising the amino acid sequence of SEQ ID NO: 12 or 13, or having an amino acid sequence of at least 80% sequence identity to SEQ ID NO: 12 (Ig1) or SEQ ID NO: 13 (Ig2), or at least 80% sequence identity to amino acids 44-215 of SEQ ID NO: 10. In some embodiments, the nucleic acid sequence does not comprise introns.

A TMIGD1 agonist comprising a polypeptide comprising the amino acid sequence of SEQ ID NO: 9 (CYR61) or having an amino acid sequence of at least 80% sequence identity to SEQ ID NO: 9.

Another aspect of the technology described herein relates to a TMIGD1 agonist comprising a nucleic acid encoding a polypeptide comprising the sequence of SEQ ID NO: 9 (CYR61) or a polypeptide having an amino acid sequence of at least 80% sequence identity to SEQ ID NO: 9.

Another aspect of the technology described herein relates to a method of treating kidney cancer in a subject in need thereof, the method comprising administering a treatment for kidney cancer to a subject determined to have a decreased level of TMIGD1.

Another aspect of the technology described herein relates to a method of diagnosing kidney cancer in a subject, the method comprising determining the level of TMIGD1 in the subject, and diagnosing the subject as having kidney cancer if the level is decreased relative to a reference level. In such embodiments, the kidney cancer is renal cell carcinoma (RCC). In some embodiments, the level of TMIGD1 is the level of TMIGD1 in a kidney biopsy sample, blood sample or urine sample. In some embodiments, a TMIGD1 agonist administered to the subject if the subjects is diagnosed as having kidney cancer, e.g., by administering a TMIGD1 agonist as disclosed herein, such as, but not limited to, an antibody reagent (e.g., activating anti-TMIGD1 antibody or antigen-binding fragment thereof); a TMIGD1 polypeptide; and a nucleic acid encoding a TMIGD1 polypeptide.

In alternative embodiments, a TMIGD1 agonist is a polypeptide comprising the sequence of SEQ ID NO: 9 (CYR61) or a polypeptide having at least 80% sequence identity to SEQ ID NO: 9 is administered to a subject if the subjects is diagnosed as haying kidney cancer.

### BRIEF DESCRIPTION OF THE DRAWINGS

FIGS. 1A-1C demonstrate the identification and characterization of TMIGD1. FIG. 1A depicts the predicted structure of human TMIGD1 mRNA. Exons are not drawn to scale. Shown are the alignment of human TMIGD1 (SEQ ID NO: 1) with mouse TMIGD1 (SEQ ID NO: 2) amino acid sequences (top panel), the human TMIGD1 amino acid sequence showing the signal sequence (visualized in blue; aa 1-29), Ig1 domain (referred to as IgI; aa 44-123 of SEQ ID NO: 1), the Ig2 domain (referred to as IgII; aa 124-215 of SEQ ID NO: 1) and the transmembrane domain (underlined; aa 219-241 of SEQ ID NO: 1) and the c-terminal cytoplasmic domain (aa 242-262 of SEQ ID NO: 1) (middle panel); and the predicted 3D structure of immunoglobulin Ig1 and Ig2 domains of the extracellular region of TMIGD1 (bottom panel). FIG. 1B depicts blots of whole cell lysates from HEK-293 cells expressing empty retroviral vector (pMSCV) or c-Myc-tag-TMIGD1 blotted with anti-c-Myc antibody or anti-TMIGD-1 antibody. Pre-incubation of anti-TMIGD1 antibody with the TMIGD1 peptide blocks the recognition of TMIGD1. Whole cell lysate was treated with the vehicle or PNGase F and blotted with anti-TMIGD1 antibody. The whole cell lysates were also blotted for PLCγ1 or HSP70 for loading control. FIG. 1C depicts blots of HEK-293 cells expressing c-Myc-TMIGD1 subjected to cell surface biotinylation. TMIGD1 was biotinylated.

FIGS. 2A-2D demonstrate that TMIGD1 promotes trans-epithelial electrical resistance (TEER) of epithelial cells. FIG. 2A depicts a graph of fully confluent HEK-293 cells expressing TMIGD1 or control empty vector seeded on the collagen coated trans-wells (4 trans-well/group) and electrical resistance was measured at day zero and five. FIG. 2B depicts a graph of fully confluent HEK-293 cells expressing TMIGD1 or control vector seeded on the collagen-coated trans-wells (4 trans-well/group) and subjected to permeability assay using fluorescently-labelled dextran. Permeability was measured at 30, 60 and 120 minutes. FIG. 2C depicts a graph of HK2 cells expressing control shRNA or TMIGD1 shRNA seeded on the collagen-coated trans-wells (4 trans-well/group) and electrical resistance was measured as FIG. 2A. The silencing effect of TMIGD1 shRNA on the expression of TMIGD1 in HK2 cells is shown. FIG. 2D depicts whole cell lysates of FIG. 2C blotted for TMIGD1 and loading control, Hsp70. Data are representative of two independent experiments.

FIGS. 3A-3C demonstrate that TMIGD1 expression in HEK-293 cells regulates cell proliferation and cell motility. FIG. 3A depicts a graph of HEK-293 cells expressing TMIGD1 or empty vector seeded on 24-well plate (quadruple well/group) and viability of cells was evaluated up to three days using MTT assay. The experiment is representative of three independent experiments. FIG. 3B depicts a graph of HEK-293 cells expressing TMIGD1 or empty vector seeded on Boyden chamber (4 wells/group) and migration of cells was evaluated after 12 hours. The experiment is representative of two independent experiments. FIG. 3C depicts a graph of HEK-293 cells expressing empty vector or TMIGD1 prepared for traction force microscopy (TFM) as described in the methods section of Example 1. Brightfield microscopic images and corresponding calculated traction vector maps for representative cells are shown. Root mean square (RMS) traction calculated for each cell is indicated in the upper right of each traction vector map. Traction vector maps show individual calculated traction vectors at each point of the computational grid that indicate direction and magnitude, as well as a color-coded “heat map” corresponding to traction magnitude. Individual cells were selected and imaged, and root mean square (RMS) traction values were calculated and shown in the graph.

FIGS. 4A-4E demonstrate that TMIGD1 protects kidney cells from hydrogen peroxide and serum withdrawal induced cell injury. FIG. 4A depicts a graph of HK2 cells expressing control shRNA or TMIGD1 shRNA seeded in 24-well plates (4 wells/group) in DMEM medium containing 10% FBS. After 12 hours, serum-containing medium was replaced with serum-free medium plus hydrogen peroxide (0.1mM). Cells were incubated for additional 8 hours and cell viability was measured by MTT assay. The result is representative of tree independent experiments. *p<0.05 compared to cells expressing control shRNA. FIG. 4B depicts a graph of HK2 Cells expressing control shRNA or TMIGD1 shRNA treated with hydrogen peroxide (0.1 mM) for 8 hours, stained with Annexin V-FITC and analyzed by FACS flow cytometer. FIG. 4C depicts a blot of expression of TMIGD1 in HK2 cells expressing control shRNA or TMIGD1 shRNA. FIG. 4D depicts a graph of HEK-293 cells expressing TMIGD1 or control vector seeded in 24-well plates (4/group) in DMEM medium plus increasing concentration of hydrogen peroxide as indicated. Cell survival was evaluated as panel A. *p<0.05 compared to control cells expressing empty vector. FIG. 4E depicts a graph of HEK-293 cells expressing control vector or TMIGD1 treated with hydrogen peroxide as outlined in FIG. 4B, stained with Annexin V-FITC and analyzed by FACS flow cytometer.

FIGS. 5A-5D demonstrate that oxidative stress promotes downregulation and ubiquitination of TMIGD1. FIG. 5A depicts the results of HK2 cells treated with different concentrations of hydrogen peroxide and whole cell lysates was subjected to western blot analysis using anti-TMIGD1 antibody and loading control Hsp70. Graph is representative of three independent experiments. FIG. 5B depicts the results of HK2 cells treated with hydrogen peroxide in the absence or presence of proteosome inhibitor, Bortezomib. Cells were lysed and whole cell lysates was blotted for TMIGD1 and loading control, Hsp70. Graph is representative of two independent experiments. FIG. 5C is a blot of HK2 cells incubated in serum-free medium or medium with hydrogen peroxide, lysed and subjected to immunoprecipitation using anti-TMIGD1 antibody followed by western blot using anti-ubiquitin (anti-Ub) antibody. Whole cell lysates were also blotted for TMIGD1 and loading control, Hsp70. FIG. 5D depicts a graph of HK2 cells seeded in 24-well plates overnight in 10% FBS followed by incubation of cells in serum-free medium (vehicle), serum-free medium plus hydrogen peroxide or hydrogen peroxide plus or minus proteosome inhibitor, Bortezomib for eight hours. Viability of cells was evaluated with MTT assay. The result is the representative of three independent experiments. *p<0.05 compared to control cells treated hydrogen peroxide only.

FIGS. 6A-6B demonstrate that TMIGD1 expression is downregulated in mouse models of kidney Ischemia reperfusion and DOCA salt uninephrectomy. FIG. 6A depicts a graph of total mRNA isolated from ischemia reperfusion, control mouse kidneys, and subjected to qPCR analysis. TMIGD1 mRNA expression normalized to 18S mRNA and the fold change in ischemia reperfusion kidneys as compared to control kidneys is shown. Means±SD of data is shown. N=3 for control, N=6 for post IR kidney. FIG. 6B depicts total mRNA isolated from DOCA salt uninephrectomy, control mouse kidneys, and subjected to qPCR analysis. TMIGD1 mRNA expression normalized to 18S mRNA and the fold change in DOCA salt uninephrectomy kidneys compared to control is shown. Means±SD of data is shown. N=3 for control, N=3 for DOCA salt uninephrectomy.

FIGS. 7A-7B demonstrate that TMIGD1 is conserved among species. FIG. 7A depicts an alignment of IGPR-1 (SEQ ID NO: 3) with TMIGD1 (SEQ ID NO: 4), which shows overall 31% similarity between IGPR-1 and TMIGD1. FIG. 7B depicts a schematic of the phylogenetic tree of TMIGD1 and was generated using SUPERFAMILY annotation program.

FIG. 8 demonstrates that PNGase F treatment reduces molecular weight of TMIGD1: Whole cell lysate was treated with the vehicle or PNGase-F and blotted with anti-TMIGD1 antibody. The whole cell lysates was also blotted for HSP70 as a loading control.

FIGS. 9A-9B demonstrate that TMIGD1 is expressed in mouse kidney proximal epithelial cells. FIG. 9A depicts a blot of proteins extracted from human kidney or human kidney cell line, HK2 cells blotted for TMIGD1. FIG. 9B depicts a blot of cell lysates derived from various tumor cell lines blotted for TMIGD1 or loading control, PLCγ1.

FIG. 10 demonstrates that the extracellular domain of TMIGD1 promotes homophilic dimerization and cell-cell interaction. Recombinant TMIGD1 encompassing the extracellular domain of TMIGD1 (GST-E-TMIGD1) fused to GST was used to demonstrate the binding of extracellular domain of TMIGD1 to full length TMIGD-1 expressed in HEK-293 cells. Cell lysates derived from HEK-293 cells expressing TMIGD1 or empty vector were subjected to GST pull-down assay followed by western blot using anti-TMIGD1 antibody. Also shown is the schematic of TMIGD1 and GST-E-TMIGD1. WCL, whole cell lysate.

FIGS. 11A-11B demonstrate that TMIGD1 regulates survival of cells in response to nutrient starvation. FIG. 11A is a graph of HK2 cells expressing control shRNA or TMIGD1 shRNA incubated in low glucose (5mM) and serum-free medium for overnight (quadruple wells/group) and cell survival was measured by MTT assay. The result is the representative of three independent experiments. FIG. 11B depicts a graph of HEK-293 cells expressing TMIGD1 or control vector plated in 24-well plates in quadruple in low glucose (5 mM) and serum-free medium for indicated days and cell survival was measured with MTT assay. The result is the representative of three independent experiments. *p<0.05 compared to control cells expressing empty vector at day 3.

FIGS. 12A-12B depict the identification of TMIGD1 as a kidney-specific gene. FIG. 12A depicts a graph of expression of TMIGD1 in a panel of human organs/tissues consisting of ovary, heart, vein, kidney, lung, liver, brain, pancreas, bone marrow and skin using quantitative PCR (qPCR). FIG. 12B depicts a blot of expression of TMIGD1 in the indicated tissues.

FIG. 13 depicts a table demonstrating that TMIGD1 expression is downregulated in human renal tumors.

FIGS. 14A-14C demonstrate that re-expression of TMIGD1 in 786-O cells inhibits cell proliferation and migration. The effect of re-expression of TMIGD1 in RCC cell line, 786-0 was analyzed. MTT (FIG. 14A) and BrdU (FIG. 14B) assays were used to measure the effect of TMIGD1 in proliferation of 786-O cells. FIG. 14C depicts a graph demonstrating that re-expression of TMIGD1 in 786-O cells also inhibited tumor cell migration.

FIGS. 15A-15C demonstrate that TMIGD1 expression is regulated by VHL. FIG. 15A depicts a graph of TMIGD1 mRNA levels in a panel of RCC tumor cell lines. FIG. 15B depicts a blot of TMIGD1 protein levels in a panel of RCC tumor cell lines. FIG. 15C depicts expression of TMIGD1 in two pVHL negative cell lines and those same lines with the addition of functional pVHL.

FIGS. 16A-16B demonstrate identification of CYR61 as a putative ligand for TMIGD1. FIG. 16A depicts binding of purified recombinant GST-TMIGD1 fusion protein attached to glutathione beads incubated with cell lysates of human colon tumor cell line, RKO and GST-TMIGD1-associated proteins and resolved on SDS-PAGE gel. Protein bands were visualized by coomassie blue and protein bands were excised and subjected mass spectrometry analysis (FIG. 16B). FIG. 16B depicts the sequence of CYR61 (SEQ ID NO: 9). FIG. 16C depicts in vitro binding assay validating the binding of CYR61 to TMIGD1.

### DETAILED DESCRIPTION

The inventors have discovered that TMIGD1 is an important regulator of cellular proliferation and survival. Suppression of TMIGD1 levels and/or activity leads to uncontrolled proliferation and low rates of survival when cellular injury occurs. Notably, such activity is directly opposed to the activity observed for the nearest-related protein, IGRP1/TMIGD2, which promotes angiogenesis and cell migration.

### Agonists of TMIGD1

As used herein, “TMIGD1” or “Transmembrane and immunoglobulin domain containing 1” is an Ig-CAM adhesion molecule expressed in renal tubular epithelial cells. Sequences are known for TMIGD1 in a number of species, e.g., human TMIGD1 (NCBI Gene ID NO: 388364) mRNA (e.g., NCBI Ref Seq: NM_206832.2 and NM_001319942.1) and polypeptide (e.g., NCBI Ref Seqs: NP_996663.1 (SEQ ID NO: 10) and NP_001306871.1 (SEQ ID NO: 11)) sequences.

As used herein, the term “agonist” refers to an agent which increases the expression and/or activity of the target by at least 10% or more, e.g. by 10% or more, 50% or more, 100% or more, 200% or more, 500% or more, or 1000% or more as compared to in the absence of the agonist. The efficacy of an agonist of, for example, TMIGD1, e.g. its ability to increase the level and/or activity of TMIGD1 can be determined, e.g. by measuring the level of an expression product of TMIGD1 and/or the activity of TMIGD1. Methods for measuring the level of a given mRNA and/or polypeptide are known to one of skill in the art, e.g. RTPCR with primers can be used to determine the level of RNA, and Western blotting with an antibody (e.g. an anti-TMIGD1 antibody) can be used to determine the level of a polypeptide. The activity of TMIGD1 can be determine according to a proliferation or survival assay as described below herein, e.g., wherein increased TMIGD1 activity is evidenced by decreased proliferation of cancer cells and/or decreased survival of injured cells.

### Polypeptide TMIGD1 Agonists

Non-limiting examples of agonists of TMIGD1 can include TMIGD1 polypeptides or fragments thereof and nucleic acids encoding a TMIGD1 polypeptide or variants thereof. In some embodiments, the agonist of TMIGD1 can be a TMIGD1 polypeptide, e.g., a TMIGD1 polypeptide having an amino acid sequence at least 80%, or at least 85% or at least 90%, or at least 95% or at least 97%, or at least 98%, or at least 99% identical to SEQ ID NO: 10 or 11. In some embodiments, the agonist of TMIGD1 can be a TMIGD1 polypeptide comprising the Ig1 and/or Ig2 domain of TMIGD1, e.g., a TMIGD1 polypeptide having an amino acid sequence at least 80%, or at least 85% or at least 90%, or at least 95% or at least 97%, or at least 98%, or at least 99% identical to SEQ ID NO: 12 or 13, or to amino acids 44-215 of SEQ ID NO: 10.

In some embodiments, the agonist of TMIGD1 can be an engineered and/or recombinant polypeptide. In some embodiments, the agonist of TMIGD1 can be a nucleic acid encoding a TMIGD1 polypeptide or a functional fragment thereof, for example, a TMIGD1 polypeptide having an amino acid sequence at least 80%, or at least 85% or at least 90%, or at least 95% or at least 97%, or at least 98%, or at least 99% identical to SEQ ID NO: 10-13, or amino acids 44-215 of SEQ ID NO: 10. In some embodiments, the agonist of TMIGD1 can be an engineered and/or recombinant polypeptide.

In some embodiments of any of the aspects described herein, the nucleic acid can be comprised by a vector, e.g., an expression vector.

### Anti-TMIGD1 Antibody TMIGD1 Agonists

In some embodiments, TMIGD1 agonits are antibody reagents, or antibody binding-fragments. Non-limiting examples of TMIGD1 agonists can include an antibody reagent (e.g. an activating antibody specific for TMIGD1); a TMIGD1 polypeptide; and a nucleic acid encoding a TMIGD1 polypeptide.

When antibody reagents, antibodies, or antigen-binding fragments thereof are used in activating target activity and/or expression, it is understood that the antibody or antigen-binding fragment thereof is an “activating” antibody or an antibody “agonist,” i.e. , it is one that is one which increases or potentiates one or more biological activities of the antigen(s) it binds. For example, it increases or promotes biological activity of the target upon binding, e.g., an activating antibody can bind a target and promote or increase the ability of the target to, e.g. interact with the cytoskeleton and/or signal its binding with a ligand. In certain embodiments, the agonist antibodies or antigen-binding fragments thereof, upon binding to their target antigen, mimic the effect of endogenous ligand binding to the target antigen(s).

Antibody reagents for use in the methods, compositions and kits as disclosed herein can be a polyclonal antibody or a monoclonal antibody, or an antigen-binding fragment thereof. In some embodiments, the antibody is a human monoclonal antibody, or a humanized monoclonal antibody or a chimeric antibody or antigen-binding fragments thereof.

In some embodiments, an antibody reagent for use in the methods, compositions and kits as disclosed herein specifically binds to the extracellular domain of TMIGD1, e.g., to any epitope, or a combination of epitopes, located in the region of amino acids 29-215 of SEQ ID NO: 10. In some embodiments, an antibody reagent for use in the methods, compositions and kits as disclosed herein specifically binds to an epitope within the Ig domain 1 (Ig1) corresponding to SEQ ID NO: 12. In some embodiments, an antibody reagent for use in the methods, compositions and kits as disclosed herein specifically binds to an epitope within the Ig domain 2 (Ig2) corresponding to SEQ ID NO: 13. In some embodiments, an antibody reagent for use in the methods, compositions and kits as disclosed herein specifically binds to an epitope spanning Ig1 and Ig2. In some embodiments, an antibody is a biospecific antibody which specifically binds to one or more epitopes located within the Ig1 or Ig2 regions, or one or more epitopes located within the Ig1 and Ig2 regions.

In some embodiments, an agonist anti-TMIGD1 antibody binds to at least one amino acid in the following sequence: V LTVNGKTENY ILD (SEQ ID NO: 15), and/or at least one amino acid in the following sequence: TTPGSQA (SEQ ID NO: 16), and/or at least one amino acid in the following sequence: SLICAVQNHT (SEQ ID NO: 17), and/or at least one amino acid in the following sequence: REEELLWYRE (SEQ ID NO: 18) and/or at least one amino acid in the following sequence: EGRVDLKSGN (SEQ ID NO: 19) and/or at least one amino acid in the following sequence: KINSSSVCVS (SEQ ID NO: 20) and/or at least one amino acid in the following sequence: SISENDNGIS (SEQ ID NO: 21) and/or at least one amino acid in the following sequence: FTCRLGRDQS (SEQ ID NO: 22), and/or at least one amino acid in the following sequence: VSVSVVLNVTFPP (SEQ ID NO: 23). In some embodiments, an agonist anti-TMIGD1 antibody binds to at least one amino acid in the following sequence: LLSGNDF QTVEEGSNVK (SEQ ID NO: 24), and/or at least one amino acid in the following sequence: LVCNVKANPQ (SEQ ID NO: 25);and/or at least one amino acid in the following sequence: AQMMWYKNSS (SEQ ID NO: 26) ; and/or at least one amino acid in the following sequence: LLDLEKSRHQ (SEQ ID NO: 27), and/or at least one amino acid in the following sequence: IQQTSESFQL (SEQ ID NO: 28), and/or at least one amino acid in the following sequence: SITKVEKPDN (SEQ ID NO: 29) and/or at least one amino acid in the following sequence: GTYSCIAKSS (SEQ ID NO: 30), and/or at least one amino acid in the following sequence: LKTESLDFHL (SEQ ID NO: 31) and/or at least one amino acid in the following sequence: IVKDKTVG (SEQ ID NO: 32).

Agonist or activating antibodies as disclosed herein can be generated by methods commonly known in the art. In some embodiments, the entire extracellular domain of TMIGD1 is used to generate an antibody, for example, a polypeptide comprising amino acids 29-215 of SEQ ID NO: 10 is used to generate antibodies or antigen-binding fragments. In some embodiments, the Ig1 (SEQ ID NO: 12) and/or the Ig2 (SEQ ID NO: 13) can be used to generate an antibody.

In some embodiments, one or more polypeptides or peptides comprising 2 or more consecutive amino acids of the following sequences: V LTVNGKTENY ILD (SEQ ID NO: 15), TTPGSQA (SEQ ID NO: 16), SLICAVQNHT (SEQ ID NO: 17), REEELLWYRE (SEQ ID NO: 18) EGRVDLKSGN (SEQ ID NO: 19) KJNSSSVCVS (SEQ ID NO: 20) SISENDNGIS (SEQ ID NO: 21) FTCRLGRDQS (SEQ ID NO: 22), VSVSVVLNVTFPP (SEQ ID NO: 23) can be used to generate an agonist anti-TMIGD1 antibody for use in the methods and compositions as disclosed herein. In some embodiments, one or more polypeptides or peptides comprising 2 or more consecutive amino acids of the following sequences: LLSGNDF QTVEEGSNVK (SEQ ID NO: 24), LVCNVKANPQ (SEQ ID NO: 25); AQMMWYKNSS (SEQ ID NO: 26) ; LLDLEKSRHQ (SEQ ID NO: 27), IQQTSESFQL (SEQ ID NO: 28), SITKVEKPDN (SEQ ID NO: 29); GTYSCIAKSS (SEQ ID NO: 30), LKTESLDFHL (SEQ ID NO: 31) or IVKDKTVG (SEQ ID NO: 32) can be used to generate an agonist anti-TMIGD1 antibody for use in the methods and compositions as disclosed herein.

In some embodiments, an antibody reagent for use in the methods, compositions and kits as disclosed herein is an activating antibody, or antibody agonist of TMIGD1 which specifically binds to the extracellular domain of TMIGD1 which is the binding site for CYR61, e.g., an antibody, or antibody agonist of TMIGD1 specifically binds to a region in Ig1 and/or Ig2 which binds to CYCR61.

In some embodiments, an activating antibody, or antibody agonist of TMIGD1 for use in the methods, compositions and kits as disclosed herein functions to promote, or stabilize dimerization of TMIGD1 polypeptides, e.g., formation of cis-dimers of TMIGD1. In some embodiments, an activating antibody, or antibody agonist of TMIGD1 functions to promote, or stabilize dimerization of TMIGD1 polypeptides, e.g., formation of trans-dimers of TMIGD1 polypeptides present on different kidney epithelial cells.

In all aspects as disclosed herein, the present application an activating antibody, or antibody agonist of TMIGD1 for use in the methods, compositions and kits as disclosed herein can be assessed for activating ability of TMIGD1, for example using in vitro and in vivo assays to assess cell survival of kidney cells in response to oxidative stress, or oxygen deprivation, as well as using in vivo models of chronic kidney disease (CKD) using a hypertensive mouse model or acute renal ischemia reperfusion (IR) model as disclosed herein in the Examples.

In some embodiments, an activating antibody, or antibody agonist of TMIGD1 for use in the methods, compositions and kits as disclosed herein blocks ubiqutination of TMIGD1 in conditions of oxidative stress or chronic kidney disease (CKD) or Acute kidney injury (AKI). Such an antibody reagent can be assessed by an ordinary skilled artisan, e.g., assessing ubiqutination of TMIGD1 in the presence of an oxidizing agent or oxygen deprivation conditions (e.g., as disclosed in the Examples and FIG. 5C).

In some embodiments, an activating antibody, or antibody agonist of TMIGD1 for use in the methods, compositions and kits as disclosed herein blocks the binding of CYR61 to the extracellular domain of TMIGD1. Such an antibody reagent can be assessed by an ordinary skilled artisan, e.g., assessing binding of CYR61to a GST-TMIGD1 fusion protein comprising the extracellular Ig1 and/or Ig2 domains of TMIGD1 in an in vitro binding assay as disclosed herein in the Example 6 (see, e.g., FIGS. 16B and 16C).

In some embodiments of any of the aspects described herein, the TMIGD1 agonist can be a polypeptide comprising the sequence of SEQ ID NO: 9 (CYR61). In some embodiments of any of the aspects described herein, the TMIGD1 agonist can be a polypeptide consisting essentially of the sequence of SEQ ID NO: 9 (CYR61). In some embodiments of any of the aspects described herein, the TMIGD1 agonist can be a polypeptide consisting of the sequence of SEQ ID NO: 9 (CYR61). In some embodiments, the agonist of TMIGD1 can be a CYR61 polypeptide or a variant thereof, e.g., a CYR61 polypeptide having an amino acid sequence at least 80%, or at least 85% or at least 90%, or at least 95% or at least 97%, or at least 98%, or at least 99% identical to SEQ ID NO: 9.

In some embodiments, the agonist of TMIGD1 can be a CYR61 polypeptide or a variant thereof, e.g., a CYR61 polypeptide having an amino acid sequence comprising at least the VWC domain of CYR61, or at least V2 in the VWC domain of CYR61.

In some embodiments of any of the aspects described herein, the TMIGD1 agonist can be a nucleic acid encoding a polypeptide comprising the sequence of SEQ ID NO: 9 (CYR61). In some embodiments of any of the aspects described herein, the TMIGD1 agonist can be a nucleic acid encoding a polypeptide consisting essentially of the sequence of SEQ ID NO: 9 (CYR61). In some embodiments of any of the aspects described herein, the TMIGD1 agonist can be a nucleic acid encoding a polypeptide consisting of the sequence of SEQ ID NO: 9 (CYR61). In some embodiments, the agonist of TMIGD1 can be a nucleic acid encoding a CYR61 polypeptide or a functional fragment thereof, for example, a CYR61 polypeptide having an amino acid sequence at least 80%, or at least 85% or at least 90%, or at least 95% or at least 97%, or at least 98%, or at least 99% identical to SEQ ID NO: 9, or encoded by the nucleic acid sequence of SEQ ID NO: 14.

In some embodiments, a TMIGD1 agonist can be a polypeptide comprising one or more domains of the CYR61 protein, e.g., can comprise one or more of the Insulin-like Growth factor Binding protein (iGFBP) domain, the Willebrand factor type C repeat (VWC) domain, the thrombospondin type 1 repeat (TSP-1) domain or the carboxy-ternunus (CT) homology domain. In some embodiments, a TMIGD1 agonist can be a polypeptide comprising at least one or more of the V2 region in the VWC domain, the T1 region in the TSP-1 domain, and H1 or H2 in the CT domain, which are disclosed in Leu, Chen et al., 2004, JBC 279 (42); 44177-44187, which is incorporated herein in its entirety by reference. It has been reported that CYR61 binds to ICAM1, and negatively regulates and leads to the downregulation of ICAM1 and VCA M1 in TNF-stimulated endothelial cells (See, Ringo et al., Extracellular Matrix protein CCN1 (CYR61) negatively regulated endothelial cell adhesion molecules, PhD Thesis, University of Rochester, 2015) Additionally, Ringo reports that the V2 binding site in the VWC domain of CYC61 binds to ICAM1 (which comprises 5 Ig-like extracellular domains which are important in self-assembly of ICAM1 into a dimer). Accordingly, in some embodiments, a TMIGD1 agonist for use in the methods and compositions as disclosed herein can be an antibody reagent which binds to the Ig1 domain, or Ig2 domain, or Ig1 and Ig2 domain of TMIGD1 and prevents CYR61 polypeptide from binding. In other embodiments, TMIGD1 agonist for use in the methods and compositions as disclosed herein can be a polypeptide or peptide comprising a fragment of at least 10, or at least 20, or at least 30 amino acids of TMIGD1 of SEQ ID NO: 44-215 which binds to the V2 in the VWC domain of CYR61 polypeptide, and/or prevents CYR61 from binding to the extracellular domain of TMIGD1.

### Subject Amenable to Treatment With TMIGD1 Agonists

As disclosed herein, aspects of the technology described herein relate to the treatment of a subject with kidney disease or injury, or kidney cancer.

As described herein, “kidney disease” refers to the deterioration of kidney function such that the kidneys fail to adequately filter toxins and/or waste products from the blood and has a wide range of etiology, including chronic or acute diseases and inflammatory and non-inflammatory diseases. While Acute Kidney Disease or injury (AKI) refers to the rapid loss of kidney function, Chronic Kidney Disease (CKD) refers to the progressive loss in renal function over a period of time. Kidney function can be determined based on any of several measures including: lowered glomerular filtration rate (GFR), increased serum creatinine concentrations, elevated albumin excretion rate or elevated albumin to creatinine concentration in the urine.

As used herein, “kidney injury” includes any injury to the proximal tubule of the kidney and includes, but is not limited to, acute kidney injury (AKI), chronic kidney disease (CKD) and kidney fibrosis.

As used herein, “acute kidney injury”, also known as “AKI” or “acute renal failure (ARF)” or “acute kidney failure”, refers to a disease or condition where a rapid loss of renal function occurs due to damage to the kidneys, resulting in retention of nitrogenous (urea and creatinine) and non-nitrogenous waste products that are normally excreted by the kidney. Depending on the severity and duration of the renal dysfunction, this accumulation is accompanied by metabolic disturbances, such as metabolic acidosis (acidification of the blood) and hyperkalaemia (elevated potassium levels), changes in body fluid balance, and effects on many other organ systems. It can be characterized by oliguria or anuria (decrease or cessation of urine production), although nonoliguric ARF may occur. Acute kidney injury may be a consequence of various causes including a) pre-renal (causes in the blood supply), which includes, but is not limited to, hypovolemia or decreased blood volume, usually from shock or dehydration and fluid loss or excessive diuretics use; hepatorenal syndrome, in which renal perfusion is compromised in liver failure; vascular problems, such as atheroembolic disease and renal vein thrombosis, which can occur as a complication of nephrotic syndrome; infection, usually sepsis, and systemic inflammation due to infection; severe burns; sequestration due to pericarditis and pancreatitis; and hypotension due to antihypertensives and vasodilators; b) intrinsic renal damage, which includes, but is not limited to, toxins or medication (e.g. some NSAIDs, aminoglycoside antibiotics, iodinated contrast, lithium, phosphate nephropathy due to bowel preparation for colonoscopy with sodium phosphates); rhabdomyolysis or breakdown of muscle tissue, where the resultant release of myoglobin in the blood affects the kidney, which can also be caused by injury (especially crush injury and extensive blunt trauma), statins, stimulants and some other drugs; hemolysis or breakdown of red blood cells, which can be caused by various conditions such as sickle-cell disease, and lupus erythematosus; multiple myeloma, either due to hypercalcemia or “cast nephropathy”; acute glomerulonephritis which may be due to a variety of causes, such as anti glomerular basement membrane disease/Goodpasture's syndrome, Wegener's granulomatosis or acute lupus nephritis with systemic lupus erythematosus; and c) post-renal causes (obstructive causes in the urinary tract) which include, but are not limited to, medication interfering with normal bladder emptying (e.g. anticholinergics); benign prostatic hypertrophy or prostate cancer; kidney stones; abdominal malignancy (e.g. ovarian cancer, colorectal cancer); obstructed urinary catheter; or drugs that can cause crystalluria and drugs that can lead to myoglobinuria & cystitis.

As used herein, the term “kidney fibrosis” also known as “renal fibrosis” refers to any condition having kidney fibrosis as a symptom or cause of the condition, or a condition that can be worsened by the development of kidney fibrosis, or a condition the progression of which is linked to the progression of kidney fibrosis. Kidney fibrosis is the formation of excess fibrous connective tissue in kidney characterized by glomerulosclerosis and tubulointerstitial fibrosis. The pathogenesis of kidney fibrosis is a monotonous process that is characterized by an excessive accumulation and deposition of extracellular matrix (ECM) components (see e.g., Y. Liu, Kidney International 2006, 69, 213-217). Kidney fibrosis can be evaluated by methods including, but not limited to, histology, immunohistochemistry, Western blot, and real-time PCR for mRNA and protein expression of extracellular matrix including collagen I and alpha-smooth muscle actin, and activation of TGF beta/Smad signaling Kidney fibrosis can result from various diseases and insults to the kidneys. Examples of such diseases and insults include chronic kidney disease, metabolic syndrome, vesicoureteral reflux, tubulointerstitial renal fibrosis, diabetes (including diabetic nephropathy), and resultant glomerular nephritis (GN), including, but not limited to, focal segmental glomerulosclerosis and membranous glomerulonephritis, mesangiocapillary GN. Since kidney fibrosis is associated with loss of blood vessels, this results in secondary ischemia which can also result in glomerulare disease with loss of glomerular function. Regardless of the primary cause, insults to the kidneys may result in kidney fibrosis and the concomitant loss of kidney function. (Schena, F. and Gesualdo, L., Pathogenic Mechanisms of Diabetic Nephropathy, J. Am. Soc. Nephrol., 16: S30-33 (2005); Whaley-Connell, A., and Sower, J R., Chronic Kidney Disease and the Cardiometabolic Syndrome, J. Clin. Hypert., 8(8): 546-48 (2006)). Conditions associated with kidney fibrosis include, but are not limited to, diabetic nephropathy, chronic kidney disease, end-stage renal disease, systemic lupus erythematosis, vasculitis, IgA nephropathy, other autoimmune diseases, paraprotein diseases, diabetes. Renal Fibrosis has three stages which are inflammation reaction stage, formation of fibrosis stage and cicatricial stage respectively. Symptoms vary depending on the stage. There are no obvious symptoms in the inflammation reaction stage. In the formation stage, symptoms occur such as frequent night urine, high potassium, high blood pressure and itchy skin and so on. In the cicatricial stage, renal failure may occur.

Non-limiting examples of kidney disease that can be treated with the methods and compositions as disclosed herein can include acute kidney injury; chronic kidney disease, kidney fibrosis, renal fibrosis, glomerulosclerosis and tubulointerstitial fibrosis, diabetic nephropathy, chronic kidney disease, end-stage renal disease, systemic lupus erythematosis, vasculitis, IgA nephropathy, other autoimmune diseases, paraprotein diseases and diabetes, and the like.

Without wishing to be bound by theory, acute kidney injury (AKI) predisposes patients to the development of both chronic kidney disease and end-stage renal failure. AKI is characterized by a rapid decline in kidney function, often triggered by an ischemic or toxic insult. This clinical syndrome is associated with substantial short-term morbidity, mortality, and cost, but it had previously been assumed that patients surviving the episode made a full renal recovery. However, AKI is now appreciated to be markedly associated with increased risk of future chronic kidney disease (CKD), end-stage renal disease (ESRD) (Ishani A, et al., J Am Soc Nephrol. 2009; 20(1):223-228.; Wald R, et al., JAMA. 2009; 302(11):1179-1185.), and long-term mortality (Lafrance J P, Miller D R, J Am Soc Nephrol. 2010; 21(2): 345-352.). The population rate of AKI is increasing at greater than 7% per year, and some estimates indicate that the incidence of AKI-related ESRD is equal to the incidence of ESRD from diabetes. The mechanisms that might explain the link between AKI and future CKD/ESRD are poorly understood, but peritubular capillary loss, a known consequence of AKI (Basile D P, et al., Am J Physiol Renal Physiol. 2001; 281(5):F887-F899.), is proposed to lead to chronic hypoxia and later development of tubulointerstitial fibrosis and CKD (Kang D H, et al., J Am Soc Nephrol. 2002; 13(3):806-816; Nangaku M., J Am Soc Nephrol. 2006; 17(1):17-25).

In some embodiments, aspects of the technology described herein relate to methods and compositions for the treatment of a subject with cancer, e.g., a cancer of epithelial origin, or kidney cancer or renal cancer.

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-fonning tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “metastases” or “metastatic tumor” refers to a secondary tumor that grows separately elsewhere in the body from the primary tumor and has arisen from detached, transported cells, wherein the primary tumor is a solid tumor. The primary tumor, as used herein, refers to a tumor that originated in the location or organ in which it is present and did not metastasize to that location from another location. As used herein, a “malignant tumor” is one having the properties of invasion and metastasis and showing a high degree of anaplasia Anaplasia is the reversion of cells to an immature or a less differentiated form, and it occurs in most malignant tumors.

As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

A subject that has a cancer or a tumor is a subject having objectively measurable cancer cells present in the subject's body. Included in this definition are malignant, actively proliferative cancers, as well as potentially dormant tumors or micrometastatses. Cancers which migrate from their original location and seed other vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

Kidney or renal cancer is a cancer occurring and/or originating in the kidneys. In some embodiments of any of the aspects, the kidney cancer can be renal cell carcinoma. In some embodiments of any of the aspects, the kidney cancer can be renal cell carcinoma, transitional cell carcinoma, renal pelvis carcinoma, squamous cell carcinoma, juxtaglomerular cell tumor (reninoma), angiomyolipoma, bellini duct carcinoma, clear-cell sarcoma of the kidney, mesoblastic nephroma, Wilms' tumor,mixed epithelial stromal tumor, clear cell adenocarcinoma, inverted papilloma, renal lymphoma, teratoma, carcinosarcoma, or carcinoid tumor of the renal pelvis.

In some embodiments, the methods described herein relate to treating a subject having or diagnosed as having, e.g., kidney cancer or disease with an agonist of TMIGD1. Subjects having, e.g., kidney cancer can be identified by a physician using current methods of diagnosing kidney cancer. Symptoms and/or complications of kidney cancer, e.g., Renal cell carcinoma (RCC), which characterize these conditions and aid in diagnosis are well known in the art and include but are not limited to, haematuria, pain in the flank, an abdominal mass, malaise, weight loss, anaemia, erythocytosis, varicocele, hypertension, hypercalcemia, fevers, and fatigue. Tests that may aid in a diagnosis of, e.g. RCC include, but are not limited to, blood and/or urine tests for renal and liver function, blood clotting time tests, an examination of the abdomen physically or by ultrasound, CT, and/or MRI. A family history of cancer, or exposure to risk factors for kidney cancer (e.g. smoking, hypertension, and obesity) can also aid in determining if a subject is likely to have kidney cancer or in making a diagnosis of kidney cancer. Another type of injury to the kidney is kidney or renal cancer.

Kidney cancer is a heterogeneous disease consisting of various subtypes with diverse generic, biochemical and morphologic features. Renal cell carcinoma (RCC) accounts for 2-3% of adult malignancies and its incidence is increasing. RCC is not a uniform disease and is subdivided into clear cell, papillary, chromophobe and oncocytoma. The most common histological subtype of RCC is conventional RCC (also referred to as clear cell RCC or ccRCC), which accounts for 70-80% of all RCC cases. Based on morphological features defined in the WHO International Histological classification of Kidney Tumors, RCC can be divided into clear cell (conventional or ccRCC) (80%), papillary RCC (chromophil) (10-15%), chromophobe RCC (5%), collecting duct RCC (<1%) and unclassified RCC (<2%) subtypes. Many patients with von Hippel Lindau (VHL) disease, an autosomal dominant genetic disorder of inherited predisposition to RCC, also develop conventional RCC and studies on this familial disease facilitated the identification of the VHL tumor suppressor gene (Latif et al., Science, 1993; 260; 1317-1320).

The term “renal cell carcinoma” and “RCC” are used interchangeably herein, refers to a tumor of the kidney. Tumors of the kidney can be malignant or benign and are the most common primary malignant kidney tumor. RCC usually begins in the cells that line the small tubes of each nephron. Renal cell tumors can grow as a single mass, and can multiple RCC tumors can develop on a single kidney or both kidneys. The term RCC encompasses different subtypes of RCC, such as, but not limited to epithelial renal cell carcinoma (RCC), clear cell (conventional), papillary RCC (chromophil), chromophobe RCC, collecting duct RCC (<1%) and unclassified RCC subtypes.

The term “clear cell RCC” refers to the most common renal neoplasm seen in adults (70% of tumors derived from tubular epithelium). Clear cell RCC can be as small as 1 cm or less and discovered incidentally, or it can be as bulky as several kilograms, and often presents pain, as a palpable mass or with hematuria, but a wide variety of paraneoplastic syndromes have been described. Clear cell RCC might be clinically silent for years and may present with symptoms of metastasis. Clear cell RCC has a characteristic gross appearance; the tumor is solid, lobulated, and yellow, with variegation due to necrosis and hemorrhage, with in some instances, the tumor circumscribed, or invade the perirenal fat or the renal vein.

The incidence of renal cell carcinoma (RCC) has steadily risen in the United States since 1970 and is currently estimated at approximately 51,000 cases per year. This increase has been observed across gender and race, increasing among black males and females by 3.9% and 4.3% per year, and white males and females by 2.3% and 3.1% per year, respectively. Typically, kidney organ confined RCC is treated with surgery and the five-year survival rate for patients presenting with Stage I disease is 95%, while the survival rate for patients with Stage II and III RCC is decreased to 70-80% and 40-60%, respectively. It is therefore reasonable to assume that early disease detection would improve overall survival in RCC patients.

RCC is a histological diverse disease, with variable and often unpredictable clinical behavior. The prognosis worsens dramatically with the onset of clinical metastasis and current regimens of systematic therapy yield only modest benefits for metastatic RCC. However, targeted therapy has opened a new set of possibilities and questions in RCC treatment. Tumor response by classical imaging criteria fails to reflect changes in tumor vessel density, tumor viability, or correlate with disease progression or even overall survival. The availability of biomarkers that reflect disease progression and severity as well as activity may therefore help guide therapy. Biomarkers that serve as surrogate markers of tumor response will expedite a large number of clinical trials in which kinase inhibitor are used in combination in patients both pre and post surgery. Treatment of patients with minimal residual disease may prove, now that effective therapies are available, to be a better approach than treatment following clinical detection. Adjuvant trials may target patients with biomarker-detected minimal residual disease after nephrectomy for the primary tumor.

Surgical resection is the mainstay of therapy for patients with localized primary tumors. However, new therapies are desperately needed for metastatic RCC, which is poorly responsive to chemotherapy and radiotherapy.

In some embodiments, the compositions and methods described herein can be administered to a subject having or diagnosed as having kidney cancer or disease. In some embodiments, the methods described herein comprise administering an effective amount of compositions described herein, e.g. an agonist of TMIGD1 to a subject in order to alleviate a symptom of a kidney cancer or disease. As used herein, “alleviating a symptom” is ameliorating any condition or symptom associated with the relevant condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of the active ingredient, e.g., an agonist of TMIGD1, needed to alleviate at least one or more symptom of the disease or disorder, and relates to a sufficient amount of pharmacological composition to provide the desired effect. The term “therapeutically effective amount” therefore refers to an amount of the active ingredient that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of an agonist of TMIGD1 which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for kidney function, among others. The dosage can be determined by a physician and adjusted, as necessary, to suit observed effects of the treatment.

In some embodiments, the technology described herein relates to a pharmaceutical composition comprising an agonist of TMIGD1 as described herein, and optionally a pharmaceutically acceptable carrier. In some embodiments, the active ingredients of the pharmaceutical composition comprise an agonist of TMIGD1 as described herein. In some embodiments, the active ingredients of the pharmaceutical composition consist essentially of an agonist of TMIGD1 as described herein. In some embodiments, the active ingredients of the pharmaceutical composition consist of an agonist of TMIGD1 as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. an agonist of TMIGD1 as described herein.

In some embodiments, the pharmaceutical composition comprising an agonist of TMIGD1 as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of an agonist of TMIGD1 as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an agonist of TMIGD1 as disclosed herein can also be incorporated into the parenteral dosage forms of the disclosure, including conventional and controlled-release parenteral dosage forms.

Pharmaceutical compositions comprising an agonist of TMIGD1 can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the agonist of TMIGD1 can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Chemg-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185 B1 ; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridincs such as bcnzodopa, carboquonc, mcturcdopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azascrinc, blcomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb™); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

In certain embodiments, an effective dose of a composition comprising an agonist of TMIGD1 as described herein can be administered to a patient once. In certain embodiments, an effective dose of a composition comprising an agonist of TMIGD1 can be administered to a patient repeatedly. For systemic administration, subjects can be administered a therapeutic amount of a composition comprising an agonist of TMIGD1, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. cell proliferation or rate of cell death by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising an agonist of TMIGD1 can be administered over a period of time, such as over a 5 minute, 10 minute, 15 minute, 20 minute, or 25 minute period.

The dosage ranges for the administration of an agonist of TMIGD1, according to the methods described herein depend upon, for example, the form of the agonist of TMIGD1, its potency, and the extent to which symptoms, markers, or indicators of a condition described herein are desired to be reduced, for example the percentage reduction desired for cell proliferation or the extent to which, for example, cell survival are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of an agonist of TMIGD1 in, e.g. the treatment of a condition described herein, or to induce a response as described herein (e.g. suppress proliferation) can be determined by the skilled clinician. However, a treatment is considered “effective treatment,” as the term is used herein, is one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. cell proliferation and/or survival rates. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response (e.g. tumor size). It is well within the ability of one skilled in the art to monitor efficacy of administration and/or treatment by measuring any one of such parameters, or any combination of parameters. Efficacy can be assessed in animal models of a condition described herein, for example treatment of cancer. When using an experimental animal model, efficacy of treatment is evidenced when a statistically significant change in a marker is observed.

In vitro and animal model assays are provided herein which allow the assessment of a given dose of an agonist of TMIGD1. By way of non-limiting example, the effects of a dose can be assessed by a cell proliferation assay. A non-limiting example of a protocol for such an assay is as follows: the dosse of, e.g, an agonist of TMIGD1 can be administered to a cancer cell line, e.g., 786-0, and proliferation measured by MTT and/or BrdU assay.

The efficacy of a given dosage combination can also be assessed in an animal model, e.g. a mouse model of kidney disease.

### TMIGD1 Agonists That Block, or Interfere With CYR61 Binding

In one aspect of any of the embodiments, described herein is an isolated activating antibody reagent which specifically binds the CYR61-binding domain of TMIGD1, thereby activating TMIGD1. As used herein, “CYR6 -binding domain of TMIGD1” refers to a region of the extracellular domain of TMIGD1.

In some embodiments, an activating antibody reagent for use in the methods, compositions and kits as disclosed herein specifically binds to the CYR61-binding domain of TMIGD1 located in the extracellular domain of TMIGD1 comprising region of amino acids 29-215 of SEQ ID NO: 10. In some embodiments, an antibody reagent for use in the methods, compositions and kits as disclosed herein specifically binds to an epitope within the Ig domain 1 (Ig1) corresponding to SEQ ID NO: 12 or to an epitope within the Ig domain 2 (Ig2) of TMIGD1 corresponding to SEQ ID NO: 13, or to one or more epitopes comprising 1 or more amino acids within SEQ ID NO: 12 and SEQ ID NO: 13. In some embodiments, an antibody reagent for use in the methods, compositions and kits as disclosed herein specifically binds to an epitope spanning Ig1 and Ig2. In some embodiments, an antibody is a biospecific antibody which specifically binds to one or more epitopes located within the Ig1 or Ig2 regions, or one or more epitopes located within the Ig1 and Ig2 regions.

An activating antibody, or antibody agonist of TMIGD1 specifically binds to the CYR61-binding domain of TMIGD1 (e.g., the extracellular domain of TMIGD1 which is the binding site for CYR61), for example, an antibody, or antibody agonist of TMIGD1 specifically binds to a region in Ig1 and/or Ig2 which binds to CYCR61.

In some embodiments of any of the aspects, a TMIGD1-activating antibody reagent as described herein hinds to at least 3 or more, consecutive or non-consecutive, amino acids in SEQ ID NO: 12 (Ig1) and/or SEQ ID NO: 13 (Ig2) of the extracellular domain of TMIGD1. In some embodiments of any of the aspects of the technology described herein, a TMIGD1-activating antibody reagent as described herein competes with, or competitively inhibits CYR61 for specific binding to TMIGD1. some embodiments of any of the aspects of the technology described herein, a. TMIGD1-activating antibody reagent as described herein blocks the interaction of the CYR61 polypeptide of SEQ ID NO: 9 with the extracellular domain of TMIGD1, e.g., blocks CYR61 binding to Ig1 (SEQ ID NO: 12) and/or Ig2 (SEQ ID NO: 13) of TMIGD1. In some embodiments, In one aspect of any of the embodiments, described herein is a pharmaceutical composition comprising an isolated activating antibody reagent which specifically binds the CYR61-binding domain of TMIGD1 and a pharmaceutically-acceptable carrier.

In one aspect of any of the embodiments, described herein is an isolated activating antibody which specifically binds the CYR61-binding domain of TMIGD1, thereby activating TMIGD1. In some embodiments of any of the aspects, a TMIGD1-activating antibody as described herein specifically binds to at least one or more amino acid in the Ig1 domain of TMIGD1 corresponding to SEQ ID NO: 12 (corresponding to amino acids 44-123 of SEQ ID NO: 10), and/or to at least one or more ammo acid in the Ig2 domain of TMIGD1 corresponding to SEQ ID NO: 13 (corresponding to amino acids 124-215 of SEQ ID NO: 10). In some embodiments of any of the aspects, a TMIGD1-activating antibody as described herein competes with CYR61 for specific binding to TMIGD1 . In one aspect of any of the embodiments, described herein is a pharmaceutical composition comprising an isolated activating antibody which specifically binds the CYR61-binding domain of TMIGD1 and a pharmaceutically-acceptable carrier.

In one aspect of any of the embodiments, described herein is a TMIGD1 agonist comprising a nucleic acid sequence encoding a TMIGD1 polypeptide. In some embodiments of any of the aspects, a nucleic acid sequence encoding a TMIGD1 polypeptide does not comprise introns. In some embodiments of any of the aspects, a TMIGD1 polypeptide comprises the sequence of SEQ ID NO 10 or 11. In some embodiments, agonists of TMIGD1 can include TMIGD1 polypeptides or fragments thereof and nucleic acids encoding a TMIGD1 polypeptide or variants thereof. In some embodiments, the agonist of TMIGD1 can be a TMIGD1 polypeptide, e.g., a TMIGD1 polypeptide having an amino acid sequence at least 80%, or at least 85% or at least 90%, or at least 95% or at least 97%, or at least 98%, or at least 99% identical to SEQ ID NO: 10 or 11. In some embodiments, the agonist of TMIGD1 can be a TMIGD1 polypeptide comprising the Ig1 and/or Ig2 domain of TMIGD1, e.g., a TMIGD1 polypeptide having an amino acid sequence at least 80%, or at least 85% or at least 90%, or at least 95% or at least 97%, or at least 98%, or at least 99% identical to SEQ ID NO: 12 or 13, or to amino acids 44-215 of SEQ ID NO: 10.

In some embodiments, the agonist of TMIGD1 can be an engineered and/or recombinant polypeptide. In some embodiments, the agonist of TMIGD1 can be a nucleic acid encoding a TMIGD1 polypeptide or a functional fragment thereof, for example, a TMIGD1 polypeptide having an amino acid sequence at least 80%, or at least 85% or at least 90%, or at least 95% or at least 97%, or at least 98%, or at least 99% identical to SEQ ID NO: 10-13, or amino acids 44-215 of SEQ ID NO: 10. In some embodiments, the agonist of TMIGD1 can be an engineered and/or recombinant polypeptide.

In one aspect of any of the embodiments, described herein is a TMIGD1 agonist comprising a polypeptide comprising the sequence of SEQ ID NO: 9 (CYR61).

In one aspect of any of the embodiments, described herein is a TMIGD1 agonist comprising a nucleic acid encoding a polypeptide comprising the sequence of SEQ ID NO: 9 (CYR61.)

In one aspect of any of the embodiments, described herein is a pharmaceutical composition comprising a TMIGD1 agonist and a pharmaceutically-acceptable carrier.

### Identifying Subjects For Treatment With TMIGD1 Agonists

As described herein, TMIGD1 levels are decreased in subjects with kidney cancer. Accordingly, in one aspect of any of the embodiments, described herein is a method of treating kidney cancer in a subject in need thereof, the method comprising administering a treatment for kidney cancer to a subject determined to have a decreased level of TMIGD1 . In one aspect of any of the embodiments, described herein is a method of diagnosing kidney cancer in a subject, the method comprising determining the level of TMIGD1 in the subject, and diagnosing the subject as having kidney cancer if the level is decreased relative to a reference level.

For example, aspects of the technology as disclosed herein relate to methods to treat a kidney injury or disease, or kidney cancer comprising identifying a subject with decreased level of TMIGD1 in a biological sample obtained from the subject, and administering to the subject an appropriate treatment for kidney cancer, and in some embodiments, the appropriate treatment is a composition comprising a TMIGD1 agonist as disclosed herein.

In some embodiments of any of the aspects, a treatment for kidney cancer can be, e.g., administration of a chemotherapeutic (e.g., nivolumab; axitinib; sunitinib; bevacizumab; sorafenib; pazopanib; everolimus; interferon-alpha; IL-2; temsirolimus; cabozantinib), radiation, surgery, percutaneous ablation therapy, and/or a TMIGD1 agonist as described herein. For example, in some embodiments, the subject can be administered a TMIGD1 agonist as described herein, alone, or in combination with (eg., before, after or during) administration of a chemotherapeutic.

In some embodiments, a subject identified to have a decreased level of TMIGD1 in a biological sample obtained from the subject is administered a composition comprising a TMIGD1 agonist as disclosed herein. In some embodiments, the composition further comprises a IGPR-1/TMIGD2 antagonist or inhibitor as disclosed in US Patent application US 2014/0227293, which is incorporated herein in its entirety by reference. In some embodiments, the subject can be administered a TMIGD1 agonist as described herein, alone, or in combination with (e.g., before, after or during) administration of a IGPR-1/TMIGD2 antagonist or inhibitor, e.g., a blocking anti-IGPR-1 antibody inhibitor as disclosed in US 2014/0227293.

In some embodiments, measurement of the level of a target and/or detection of the level or presence of a target, e.g. of an expression product (nucleic acid or polypeptide of TMIGD1) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments, a DNA sample can undergo enzymatic replication, e.g., by polymerase chain reaction (PCR).

Transformation, measurement, determining of the precence of, and/or detection of a target molecule, e.g. a DNA sequence, a mRNA, or, a polypeptide can comprise contacting a sample obtained from a subject with a reagent (e.g. a detection reagent) which is specific for the target, e.g., a target-specific reagent. In some embodiments, the target-specific reagent is detectably labeled. In some embodiments, the target-specific reagent is capable of generating a detectable signal. In some embodiments, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for the various targets described herein are commercially available and can be used for the purposes of the invention to measure protein expression levels, e.g. anti-TMIGD. Alternatively, since the amino acid sequences for the targets described herein are known and publically available at the NCBI website, one of skill in the art can raise their own antibodies against these polypeptides of interest for the purpose of the invention.

The amino acid sequences of the polypeptides described herein have been assigned NCBI accession numbers for different species such as human, mouse and rat.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments, the immunoassay can be a quantitative or a semi-quantitative immunoassay.

An immunoassay is a biochemical test that measures the concentration of a substance in a biological sample, typically a fluid sample such as urine, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent applications Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In some embodiments, the level of a target can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the gene expression products as described herein can be instead determined by determining the level of messenger RNA (mRNA) expression of the genes described herein. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNAse protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a therniostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the genes described herein have been assigned NCBI accession numbers for different species such as human, mouse and rat. For example, the human TMIGD1 mRNA is known. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofluorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

In some embodiments of any of the aspects, the level of TMIGD1 is the level of TMIGD1 in a sample obtained from the subject. In some embodiments of any of the aspects, the level of TMIGD1 is the level of TMIGD1 in a biological sample, such as, but not limited to a kidney biosy sample, blood sample, or urine sample. Blood samples include, but are not limited to, whole blood, serum or plasma. In some embodiments, the whole blood sample is further processed into serum or plasma samples. The term also includes a mixture of the above-mentioned samples.

The term “sample” or “test sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, a test sample can be a tumor cell test sample, e.g. the sample can comprise cancerous cells, cells from a tumor, and/or a tumor biopsy.

The test sample can be obtained by removing a sample from a subject, but can also be accomplished by using previously isolated samples (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the presence of a cop-gained region as described herein.

In some embodiments, the methods, assays, and systems described herein can further comprise a step of obtaining a test sample from a subject. In some embodiments, the subject can be a human subject.

A level which is less than a reference level can be a level which is less by at least about 10%, at least about 20%, at least about 50%, at least about 60%, at least about 80%, at least about 90%, or less than the reference level. In some embodiments, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments, a level which is more than a reference level can be a level which is statistically significantly greater than the reference level.

In some embodiments, the reference can be a level of the target molecule in a population of subjects who do not have or are not diagnosed as having, and/or do not exhibit signs or symptoms of a condition or state described herein (e.g., kidney cancer). In some embodiments, the reference can also be a level of expression of the target molecule in a control sample, a pooled sample of control individuals or a numeric value or range of values based on the same. In some embodiments, the reference can be the level of a target molecule in a sample obtained from the same subject at an earlier point in time, e.g., the methods described herein can be used to determine if a subject's state or condition (e.g., risk for cancer, and/or progression of cancer) is changing over time.

In some embodiments, the level of expression products of no more than 200 other genes is determined. In some embodiments, the level of expression products of no more than 100 other genes is determined. In some embodiments, the level of expression products of no more than 20 other genes is determined. In some embodiments, the level of expression products of no more than 10 other genes is determined.

In some embodiments of the foregoing aspects, the expression level of a given gene can be normalized relative to the expression level of one or more reference genes or reference proteins.

Described herein is a novel cell surface receptor, TMTGD1/IGPR-2, the characterization of its function, and the development of a polyclonal anti-IGPR-2 antibody. It is demonstrated herein that TMIGD1 regulates cell migration and cell morphology and protects renal epithelial cells from oxidative and nutrient deprivation-induced cell injury. TMIGD1/IGPR-2 is a new member of IGPR-1 family, which is described in US2014/0227293, which is incorporated herein in its entirety by reference. IGPR-1 is a cell adhesion molecule involved in angiogenesis and tumor growth and metastasis. The present data demonstrates that TMIGD1 acts to protect kidney epithelial cells from oxidative cell injury to promote cell survival, indicating a key role for TMIGD1 in kidney diseases, e.g. cancer.

Targeting TMIGD1 either by increasing its expression via gene delivery or small molecules that bind to promoter of TMIGD1 and increases its expression can be exploited for novel therapeutic in kidney diseases. As disclosed herein, an activating anti-TMIGD1 antibody can be used for the treatment of kidney disease or injury and/or kidney cancer.

As described herein, a short peptide corresponding to the external domain of TMIGD1/IGPR-2 (e.g., amino acids 41-215 of SEQ ID NO: 10) was used to generate a polyclonal anti-IGPR-2 antibody.

In some embodiments, agonist anti-TMIGD1 antibody for use in the methods and compositions as disclosed herein can be generated by methods commonly known in the art. In some embodiments, the entire extracellular domain of TMIGD1 is used to generate an antibody, for example, a polypeptide comprising amino acids 29-215 of SEQ ID NO: 10 is used to generate antibodies or antigen-binding fragments. In some embodiments, the Ig1 (SEQ ID NO: 12) and/or the Ig2 (SEQ ID NO: 13) can be used to generate an antibody.

In some embodiments, one or more polypeptides or peptides comprising 2 or more consecutive amino acids of the following sequences: V LTVNGKTENY ILD (SEQ ID NO: 15), TTPGSQA (SEQ ID NO: 16), SLICAVQNHT (SEQ ID NO: 17), REEELLWYRE (SEQ ID NO: 18) EGRVDLKSGN (SEQ ID NO: 19) KINSSSVCVS (SEQ ID NO: 20) SISENDNGIS (SEQ ID NO: 21) FTCRLGRDQS (SEQ ID NO: 22), VSVSVVLNVTFPP (SEQ ID NO: 23) can be used to generate an agonist anti-TMIGD1 antibody for use in the methods and compositions as disclosed herein. In some embodiments, one or more polypeptides or peptides comprising 2 or more consecutive amino acids of the following sequences: LLSGNDF QTVEEGSNVK (SEQ ID NO: 24), LVCNVKANPQ (SEQ ID NO: 25); AQMMWYKNSS (SEQ ID NO: 26) ; LLDLEKSRHQ (SEQ ID NO: 27), IQQTSESFQL (SEQ ID NO: 28), SITKVEKPDN (SEQ ID NO: 29); GTYSCIAKSS (SEQ ID NO: 30), LKTESLDFHL (SEQ ID NO: 31) or 1VKDKTVG (SEQ ID NO: 32) can be used to generate an agonist anti-TMIGD1 antibody for use in the methods and compositions as disclosed herein.

### Compositions

Aspects of the present invention relate to compositions comprising a TMIGD1 agonist as disclosed herein. In some embodiments, the TMIGD1 agonist is an activating anti-TMIGD1 antibody or antibody reagent as disclosed herein, e.g., an activating anti-TMIGD1 antibody or antigen-binding fragment thereof which binds to Ig1 and/or Ig2 of TMIGD1.

In some embodiments, the compositions, e.g., antibodies described herein can permit analysis of TMIGD1/IGPR-2 by, e.g., Western blot, Immunoprecipitation, Immunohistochemistry. In some embodiments, the antibody agents described herein detect mammalian TMIDG1, e.g., human and mouse TMIGD1.

### Kits:

Another aspect of the present disclosure is directed towards kits. The kits can comprise a suitable container comprising a TMIGD1 agonist as disclosed herein, e.g., an activating anti-TMIGD1 antibody or antibody reagent as disclosed herein, e.g., an activating anti-TMIGD1 antibody or antigen-binding fragment thereof which binds to Ig1 and/or Ig2 of TMIGD1 and a pharmaceutically acceptable carrier, and instructions for use.

Another aspect of the present invention relates to a kit comprising an anti-TMIGD1 antibody or antigen-binding fragment thereof and reagents necessary for detecting levels of TMIGD1 in a biological sample obtained from a subject. In some embodiments, the kit comprises an immunological assay, such as, but not limited to, a dip-stick assay for detecting the levels of TMIGD1 in a biological sample obtained from a subject.

The following examples are included to demonstrate preferred embodiments of the invention. It should be appreciated by those of skill in the art that the techniques disclosed in the examples which follow represent techniques discovered by the inventor(s) to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

Having generally described this invention, the same will become more readily understood by reference to the following specific examples which are included herein for purposes of illustration only and are not intended to be limiting unless otherwise specified.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of nonnal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the specific polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are also known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

### Expression of TMIGD1

Aspects of the technology as disclosed herein relate to expression of a TMIGD1 agonist from an expression vector. In some embodiments, a TMIGD1 agonist is encoded by a nucleic acid encoding a TMIGD1 polypeptide corresponding to SEQ ID NO: 10 or SEQ ID NO: 11 or a polypeptide having at least 80% sequence identity to SEQ ID NO: 10 or 11, or a functional fragment thereof, e.g., a polypeptide comprising the Ig1 domain of SEQ ID NO: 12 and/or Ig2 domain of SEQ ID NO:13 or a polypeptide having at least 80% sequence identity to SEQ ID NO: 12 or 13. In some embodiments, a TMIGD1 agonist is encoded by a nucleic acid encoding a CYR61 polypeptide corresponding to SEQ ID NO: 9 or a polypeptide having at least 80% sequence identity to SEQ ID NO: 9. In some embodiments, a TMIGD1 agonist is encoded by a nucleic acid encoding a CYR61 polypeptide comprising a portion of SEQ ID NO: 9 that binds to the TMIGD1 polypeptide and can block CYR61 from binding.

In some embodiments, the expression of a TMIGD1 agonist from an expression vector is to increase expression of TMIGD1 polypeptide to a normal level in a cell, e.g., kidney cell, where TMIGD1 levels are decreased or below a normal level.

In some embodiments, the expression of a TMIGD1 agonist from an expression vector is to overexpress the TMIGD1 polypeptide to a level above a normal level in a cell, e.g., kidney cell, where TMIGD1 polypeptide levels are normal or below a nonnal level. In such an embodiment, the level of TMIGD1 polypeptide is overexpressed by at least about 10%, or about 20%, or about 30%, or about 40%, or about 50%, or about 60%, or about 70%, or about 80%, or about 90%, or about 100%, or about 1.2-fold, or about 1.5-fold, or about 2.0-fold, or about 2.5-fold, or about 3.0-fold, or about 4.0-fold, or about 5.0-fold or more than 5-fold the level of normal TMIGD1 polypeptide expression in kidney cell.

Overexpression can be achieve by any method known in the art, and includes, but not limited to, using expression vectors with strong promoters, using enhancer as well as inserting multiple nucleic acid sequences encoding the protein to be expressed (e.g., a TMIGD1 polypeptide as described herein).

In some embodiments, a nucleic acid encoding a polypeptide as described herein (e.g. a TMIGD1 or CYR61 polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In some embodiments, an inhibitor or agonist of a given polypeptide can be an antibody reagent specific for that polypeptide. As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab')2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. The phrase can also refer to continuous or discontinuous epitopes in which the primary sequence (i.e., the amino acid sequence) is not similar but nonetheless the epitopes are still recognized by the same antibody.

The term “antibody variant” is intended to include antibodies produced in a species other than a mouse. It also includes antibodies containing post translational modifications to the linear polypeptide sequence of the antibody or fragment. It further encompasses fully human antibodies. The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized.

The term “human antibody” as used herein, is intended to include antibodies having variable and constant regions derived from human germline immunoglobulin sequences. The human antibodies of the present invention can include amino acid residues not encoded by human germline immunoglobulin sequences (e.g., mutations introduced by random or site-specific mutagenesis in vitro or by somatic mutation in viva). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, CL, CH domains (e.g., CH1, CH2, CH3), hinge, (Via, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain); genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human gennline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody can be at least about 95%, or even at least about 96%, or least about 97%, or least about 98%, or least about 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody can display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human monoclonal antibody” refers to antibodies displaying a single binding specificity which have variable and constant regions derived from human germline immunoglobulin sequences. The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in viva somatic mutagenesis) and thus the amino acid sequences of the VH and VL regions of the recombinant antibodies are sequences that, while derived from and related to human germline VH and VL sequences, can not naturally exist within the human antibody germline repertoire in vivo. As used herein, “isotype” refers to the antibody class (e.g., IgM or IgG1) that is encoded by heavy chain constant region genes.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized. In particular, the terms “specifically binds,” “specific binding affinity” (or simply “specific affinity”), and “specifically recognize,” and other related terms when used to refer to binding between a protein and an antibody, refers to a binding reaction that is determinative of the presence of the protein in the presence of a heterogeneous population of proteins and other biologics. Thus, under designated conditions, a specified antibody binds preferentially to a particular protein (e.g., TMIGD1) and does not bind in a significant amount to other proteins present in the sample. An antibody that specifically binds to a protein has an association constant of at least 10³M⁻¹ or 10⁴M⁻¹, sometimes 10⁵M⁻¹ or 10⁶M⁻¹, in other instances 10⁶M⁻¹ or 10¹⁰M⁻¹, preferably 10⁸M⁻¹ to 10⁹M⁻¹, and more preferably, about 10¹⁰M⁻¹ to 10¹¹M⁻¹ or higher. Protein-binding molecules with affinities greater than 10⁸M⁻¹ are useful in the methods of the present invention. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a given target.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be an artificial or engineered carrier, e.g., a carrier that the active ingredient would not be found to occur in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff s Clinical Oncology, 2013 Elsevier; and Fischer D S (ed): The Cancer Chemotherapy Handbook, 4th ed. St. Louis, Mosby-Year Book, 2003).

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The technology described herein is further illustrated by the following examples which in no way should be construed as being further limiting.

All references disclosed herein in the specificiaton and Examples are incorporated herein by reference in their entirety.

Some embodiments of the technology described herein can be defined according to any of the following numbered paragraphs:
1. A method of treating kidney disease or cancer in a subject in need thereof, the method comprising administering a TMIGD1 agonist to the subject.
2. The method of paragraph 1, wherein the agonist is selected from the group consisting of:
   i. an antibody reagent; a TMIGD1 polypeptide; and a nucleic acid encoding a TMIGD1 polypeptide.
3. The method of paragraph 1, wherein the agonist is a polypeptide comprising the sequence of SEQ ID NO: 9 (CYR61) or a polypeptide having at least 80% sequence identity to SEQ ID NO: 9.
4. The method of any of paragraphs 1-3, wherein the kidney cancer is renal cell carcinoma (RCC).
5. The method of any of paragraphs 1-4, wherein the kidney disease is selected from the group consisting of: acute kidney injury (ARI); chronic. kidney disease (CRD), kidney cysts, Alport Syndrome, Diabetic Nephropathy, Fabry Disease, Focal Segmental Glomerulosclerosis, Glomerulonephritis, IgA Nephropathy (Berger's Disease), Kidney Stones and Polycystic Kidney Disease (PKD).
6. The method of paragraph 2, wherein the antibody reagent is an antibody or antigen binding fragment thereof.
7. The method of paragraph 6, wherein the antibody is selected from the group consisting of: polyclonal antibody, monoclonal antibody, humanized or chimeric antibody or human monoclonal antibody.
8. The method of any of paragraphs 1 to 7, wherein the antibody reagent specifically binds to the extracellular domain of the TMIGD1 polypeptide corresponding to amino acids 41-215 of SEQ ID NO: 10.
9. The method of any of paragraphs 1 to 7, wherein the antibody reagent specifically binds to any region of: Ig1 (corresponding to amino acids 44-123 of SEQ ID NO: 10), Ig2 (corresponding to amino acids 124-215 of SEQ ID NO: 10) or Ig1 and Ig2.
10. The method of any of paragraphs 1 to 7, wherein the antibody reagent blocks the interaction or binding of CYR61 with the extracellular domain of the TMIGD1 polypeptide corresponding to amino acids 41-215 of SEQ ID NO: 10.
11. An isolated activating antibody which specifically binds the CYR61-binding domain of TMIGD1, thereby activating TMIGD1.
12. The antibody of paragraph 11, wherein the CYR61 binding domain is located in the extracellular domain of the TMIGD1 polypeptide corresponding to amino acids 41-215 of SEQ ID NO: 10.
13. The antibody of any of paragraphs 11-12, wherein the activating antibody specifically binds to at least 1 or more amino acids in any region of: Ig1 (corresponding to amino acids 44-123 of SEQ ID NO: 10), Ig2 (corresponding to amino acids 124-215 of SEQ ID NO: 10) or Ig1 and Ig2.
14. The antibody of any of paragraphs 11-13, wherein the activating antibody competes with CYR61 for specific binding to TMIGD1.
15. The antibody of any of paragraphs 11-14, wherein the activating antibody is selected from: a polyclonal antibody, a monoclonal antibody, a human antibody, a humanized antibody a chimeric antibody or antigen-binding fragments thereof.
16. A TMIGD1 agonist comprising a nucleic acid sequence encoding a TMIGD1 polypeptide.
17. The agonist of paragraph 16, wherein the nucleic acid sequence does not comprise introns.
18. The agonist of any of paragraphs 16-18, wherein the TMIGD1 polypeptide comprises the sequence of at least 80% sequence identity to SEQ ID NO: 10 or 11.
19. The agonist of any of paragraphs 16-18, wherein the TMIGD1 polypeptide comprises the amino acid sequence of at least 80% sequence identity to SEQ ID NO: 12 (Ig1) or SEQ ID NO: 13 (Ig2), or at least 80% sequence identity to amino acids 44-215 of SEQ ID NO: 10.
20. A TMIGD1 agonist comprising a polypeptide comprising the amino acid sequence of SEQ ID NO: 9 (CYR61) or having an amino acid sequence of at least 80% sequence identity to SEQ ID NO: 9.
21. A TMIGD1 agonist comprising a nucleic acid encoding a polypeptide comprising the sequence of SEQ ID NO: 9 (CYR61) or a polypeptide having an amino acid sequence of at least 80% sequence identity to SEQ ID NO: 9.
22. A method of treating kidney cancer in a subject in need thereof, the method comprising administering a treatment for kidney cancer to a subject determined to have a decreased level of TMIGD1.
23. A method of diagnosing kidney cancer in a subject, the method comprising determining the level of TMIGD1 in the subject, and diagnosing the subject as having kidney cancer if the level is decreased relative to a reference level.
24. The method of any of paragraphs 22-24, wherein the kidney cancer is renal cell carcinoma.
25. The method of any of paragraphs 15-17, wherein the level of TMIGD1 is the level of TMIGD1 in a kidney biopsy sample, blood sample or urine sample.
26. The method of paragraph 22, wherein the treatment is a TMIGD1 agonist selected from the group consisting of: an antibody reagent; a TMIGD1 polypeptide; and a nucleic acid encoding a TMIGD1 polypeptide.
27. The method of paragraph 22, wherein the treatment is a polypeptide comprising the amino acid sequence of SEQ ID NO: 9 (CYR61) or having an amino acid sequence of at least 80% sequence identity to SEQ ID NO: 9.
28. The method of paragraph 22, wherein the treatment comprises administering a composition of any of paragraphs 11-21.

### EXAMPLES

Methods and Materials:

Plasmids, shRNA and antibodies: A mouse TMIGD1 clone (cDNA clone MGC:74197, IMAGE:30311543) was purchased from Open Biosystems and subsequently was cloned as a c-myc tag in its C-terminus into retroviral pQCXIP vector via NotI and BamH1 sites. C-myc tagged TMIGD1 was further sequenced to confirm its sequence identity. Rabbit polyclonal anti-TMIGD1 antibody was made against a peptide corresponding to 20 amino acids in the extracellular domain of TMIGD1. TMIGD1 shRNA (cat #SC94146-sh) was purchased from Santa Cruz biotechnology. It consists of a pool of three to five lentiviral vector plasmids each encoding TMIGD1-specific 19-25 nt (plus hairpin) shRNAs.

Cell Lines: HEK-293 (human embryonic kidney epithelial cells) and HK2 (human kidney tubular epithelial cells) cells were grown in DMEM supplemented with 10% FBS plus antibiotics. HEK-293 cells were used to express the TMIGD1 construct. The pMSCV-puro retroviral vector was used to clone Myc-tagged TMIGD1. Viruses were produced in 293-GPG cells as described³⁴.

Cell viability and apoptosis assays: The MTT assay, which is based on the reduction of tetrazolium salt by living cells, was used to measure cell survival as described³⁵. In brief, cells (5×10⁴/well) were seeded in 24-well plates (quadruple wells/group). After overnight incubation, cells were washed with PBS, replaced with serum-free medium or other conditions as indicated in the figure legends, and subjected to MTT assay. Results were processed as recommended by the manufacturer. For FACS analysis, cells were grown in 60 cm plates, treated with hydrogen peroxide as indicated in the figure legend, subjected to staining using Annexin V-FITC Apoptosis Kit (BD Biosiences) according to the manufacturer's instructions, and analyze by FACS flow cytometer.

Cell permeability and trans-epithelial electrical resistance (TEER) assays: Cell permeability was performed as described³⁶. Briefly, HEK-293 cells expressing TMIGD1 or HK2 cells expressing TMIGD1-shRNA cells and corresponding control cells were seeded on tissue culture polycarbonate membrane filters coated with collagen I (pore size 3.0 μm) in 24-well Transwell® plates at a seeding density of 200,000 cells/well . The complete DMEM medium was added to both the donor and the acceptor compartment and incubated at 37° C. in a CO₂ incubator for at least 5 days or until a monolayer of cells was formed. FITC-Dextran (70 KD), 100 μl of 1×) was added to the each insert and incubated at 37° C. in a CO₂ incubator for the indicated time. At the end of each time point, the inserts were removed and solutions from the lower chambers were transferred into a 96-well plate and read at 485 nm excitation, 520 nm emission. For the TEER assay was cells were similarly prepared and TEER assay was performed using a Millicell® ERS meter (Millipore, Bedford, Mass., USA). The values of TEER were determined by measuring the potential difference between the two sides of the cell monolayer as recommended by the manufacturer.

Cell aggregation assay: The assay was performed as previously described¹⁷. Briefly, cells were detached from the plates with EDTA (1 mM) in phosphate-buffered saline (PBS) solution, washed twice in 10% DMEM medium and were re-suspended in DMEM. Approximately 5×10⁵ cells per 2.5 ml were incubated in a six-well plate pre-coated with 1% bovine serum albumin with gentle shaking at 37° C. for 1 h, followed by no shaking for 30 min. Cells were viewed under a light microscope, and images were taken.

Real time PCR: Total RNA was isolated using TRIzol™ reagent (Life Technologies, Carlsbad, Calif.). One microgram of total RNA, which was pooled from three mice per group was reverse transcribed using the High Capacity cDNA Reverse Transcription Kit (Life Technologies, Carlsbad, Calif.) according to the manufacturer's protocol. Gene expression was determined by quantitative PCR using the RT² SYBR Green qPCR Mastermix kit according to manufacturer's protocol. Specific PCR primers for mouse TMIGD1 (5′-GAC CCG AAT TCA GAA ACA C-3′ (SEQ ID NO: 5) and 3′-ATG CAA AAC TCT TCC CG-5′ (SEQ ID NO: 6)) were used to amplify TMIGD1. Mouse 18S PCR primers (5′ -CGC CGC TAG AGG TGA AAT TC-3′ (SEQ ID NO:7) and 3′-CCA GTC GGC ATC GTT TAT GG (SEQ ID NO: 8)) used for amplification of 18S ribosomal RNA. Data was presented as fold increase of TMIGD1 normalized to the housekeeping gene, 18S.

In Vitro GST Pull-down Assay: The extracellular domain of TMIGD1 encompassing the immunoglobulin domains was cloned into pGX2T vector and recombinant protein was prepared as described³⁷. The purified GST-fusion TMIGD1 protein subsequently was used for GST pull down assay. The assay was performed as described³⁸. Briefly, HEK-293 cells expressing TMIGD1 were grown in 10-cm plates. The plates were washed with H/S buffer and lysed in EB lysis buffer. The normalized cell lysates were incubated with equal amounts of immobilized GST fusionTMIGD1 or GST control for 3 h at 4° C. The beads were washed with phosphate buffered saline solution with protease inhibitors. The eluted proteins were boiled in sample buffer and analyzed by Western blotting using the appropriate antibody.

Bioinformatics analysis: Ensembl Genome Browser was used obtain genomic information of TMIGD1 such determining chromosomal location, coding exons, etc. SUPERFAMILY annotation program was used to generate phylogenetic tree and other structural/domain information. Swiss model 3D modeling program was used to predict 3D structure of TMIGD1.

Immunoprecipitation and Western blot: Cells were prepared and lysed as described³⁹. Briefly, cells were washed twice with H/S buffer (25 mM HEPES (pH 7.4), 150 mM NaCl, and 2 mM Na3VO4) and lysed in lysis buffer containing Triton X-100. The normalized cell lysates were immunoprecipitated as indicated in the figure legends, and immunoprecipitated proteins were subsequently subjected to Western blotting using appropriate antibody as indicated in the figure legends. Cell surface biotinylationwas performed as described¹⁷ according to the recommendations of the manufacturer.

Cell traction assay: Cell traction assay was performed as previously described⁴⁰. In brief, thin polyacrylamide gels (7.5% acrylamide and 0.2% bis-acrylamide) containing 0.5 μm diameter red fluorescent latex spheres (FluoSpheres, Invitrogen, Carlsbad, Calif.) at 0.001% concentration were polymerized between a glutaraldehyde-treated 22 mm square coverslip and an untreated 12 mm round coverslip after addition of tetramethylethylenediamine (TEMED). The round coverslips were gently removed after 30 min polymerization, and the substrate surface was chemically activated by immersing the substrate in pure hydrazine hydrate (Sigma-Aldrich, St. Louis, Mo.). Activated substrate assemblies were washed with 5% acetic acid and water for 1 hour each, immersed in poly-D-lysine (BD Biosciences, San Jose, Calif.), and washed extensively with PBS. Poly-D-lysine coated substrates were plated with cells, and incubated at 37° C. in 5% CO₂, to allow for cell attachment and spreading. Immediately prior to imaging, the cell-seeded substrate assemblies were mounted individually in 6 cm cell culture plates under standard media. Traction Force Microscopy was performed using an automated upright epifluorescence microscope (Nikon Labophot 2™, Melville, N.Y.) equipped with immersion objectives and an environmental chamber. Dell Optiplex GX280™ computer running Simple PCI™ software (Hamamatsu Corporation, Sewickley, Pa.), which was used to collect the required digital images. The substrate surface was initially scanned at low power using bright field illumination to identify and record x,y coordinate locations of fields containing candidate cells. Fourier Transform Traction Cytometry (FTTC) was performed to extract substrate displacement and traction fields, and root mean square (RMS) traction generated by each cell, using software kindly provided by Dr. Donald Ingber⁴⁰.

Animal models: DOCA salt uninephrectomy model: Ten to twelve week old 129/SVE mice were obtained from Taconic Farms. After a week of acclimatizing, surgeries were performed on mice. Mice were anesthetized by isoflurane inhalation after which right uninephrectomy was performed followed by implantation of a 21-day release DOCA salt pellet (Innovative research of America). After recovery from anesthesia, animals were housed singly in cages and fed on standard chow and given 1% saline as source of drinking water. Control animals were maintained on standard chow and tap water. For immunohistochemistry studies, mice were sacrificed at 0, 24 hours and 2 weeks after DOCA-salt uninephrectomy treatment.

Kidney Ischemia reperfusion mouse model: Six week old mice weighing 20-22 g were utilized in the ischemia-reperfusion studies. After anesthesia with isoflurane, kidneys were exposed by generating a mid-line laparotomy. The left kidney was removed. Transient Kidney ischemia was performed by clamping the right renal pedicles using a non-traumatic vascular clamp for 25 minutes, an insult that produces severe, reversible acute renal failure⁴¹. The clamp was then removed and restoration of blood flow (reperfusion) was visually confirmed. The abdominal section was closed using sutures. For histological and immunohistochemistry analysis, mice were sacrificed 48 hours post ischemia reperfusion. Left kidneys served as control kidneys. Use of mice was approved by the Institutional Animal Care and Use Committee of Boston University.

Immunohistochemistry analysis: Immunohistochemistry staining was performed as per the manufacturer's instruction using the EXPOSE Rabbit specific HRP/DAB detection IHC kit (Abeam, Cambridge, USA).

### Example 1

TMIGD1 is a Novel Adhesion Molecule that Protects Epithelial Cells From Oxidative Cell Injury

Described herein is a novel cell surface receptor, TMIGD1/IGPR-2, characterization of its function, and development of a polyclonal anti-IGPR-2 antibody. Further, it is demonstrated herein that TMIGD1 regulates cell migration and cell morphology and protects renal epithelial cells from oxidative and nutrient deprivation-induced cell injury. The present data demonstrates that TMIGD1 acts to protect kidney epithelial cells from oxidative cell injury to promote cell survival and indicates a key role for TMIGD1 in kidney diseases and cancer. Thus, TMIGD1 is a novel attractive drug target.

Targeting TMIGD1 either by increasing its expression via gene delivery or small molecules that bind to promoter of TMIGD1 and increases its expression can be exploited for novel therapeutic in kidney diseases.

Oxidative damage to renal tubular epithelial cells is a fundamental pathogenic mechanism implicated in both acute kidney injury (AKI) and chronic kidney diseases (CKD). As epithelial cell survival influences the outcome of AKI and CKD, identifying its molecular regulators can provide new insight into pathobiology and possible new therapeutic strategies for these diseases. Described herein is TMIGD1 (transmembrane and immunoglobulin containing 1), a novel adhesion molecule, which is highly conserved in humans and other species. TMIGD1 is expressed in renal tubular epithelial cells and promotes cell survival. The extracellular domain of TMIGD1 contains two putative immunoglobulin domains and mediates self-dimerization. The data provided herein demonstrate that TMIGD1 regulates trans-epithelial electric resistance and permeability of renal epithelial cells. More importantly, TMIGD1 regulates cell migration and cell morphology and protects renal epithelial cells from oxidative and nutrient deprivation-induced cell injury. Hydrogen peroxide-induced oxidative cell injury downregulates TMIGD1 expression and targets it for ubiquitination. Moreover, TMIGD1 expression is downregulated in both mouse AKI and in deoxy-corticosterone acetate (DOCA) and sodium chloride (DOCA-salt)-induced chronic hypertensive kidney disease models. Taken together, TMIGD1 as identified as a novel cell adhesion molecule expressed in kidney epithelial cells and acts to protect kidney epithelial cells from oxidative cell injury to promote cell survival.

Without wishing to be bound by theory, kidney failure occurs when the kidneys lose their ability to function due to acute or chronic diseases¹. Both acute kidney injury (AKI) and chronic kidney disease (CKD) are major kidney diseases associated with high morbidity and mortality². Though two distinct entities, emerging evidence strongly indicates close interconnection between AKI and CKD, wherein the occurrence of one strongly predicts the risk of the other^{3,4}. This interconnection also points to the presence of possible common underlying molecular mechanisms in AKI and CKD⁴. Renal tubular epithelial cells constitute the majority of renal mass and are the common damaged cell-type in both AKI and CKD^{5,6}. Hypoxia, ischemia reperfusion injury and oxidative stress damage are common pathological assaults that inflict injury on epithelial cells and the endurance of these cells strongly influences the clinical outcome^{7,8}.

Cell adhesion plays a major role in kidney injury and repair. In response to insults such as ischemia or toxins, kidney epithelial cells lose their cell-cell and cell-matrix interactions leading to loss of cell polarity, increased permeability and cell death⁹⁻¹¹. These events contribute to intraluminal aggregation of cells and proteins, causing in tubular obstruction¹²⁻¹³. The loss of cell adhesion in injured cells proceeds changes in the distribution of actin and actin-binding proteins with altered morphological and cytoskeletal changes¹⁰ which leads to reduced sodium transport and other impairments¹⁴. Kidney epithelium has a remarkable regenerative capacity post-ischemic/toxic injury. During the repair process, kidney tubular epithelial cells undergo a complex series of regenerative events such as proliferation, migration and epithelial-mesenchymal transition (EMT), leading to restoration of functional tubular epithelial cells¹⁵. Cell adhesion plays a prominent role in these regenerative processes¹⁶.

Immunoglobulin and proline rich receptor-1 (IGPR-1) is a cell adhesion molecule encoded by TMIGD2 (transmembrane and Immunoglobulin domain containing 2)¹⁷. IGPR-1/TMIGD2 is expressed in human endothelial and epithelial cells, but absent in the mouse genome. IGPR-1 is involved in angiogenesis and its activity regulates endothelial capillary tube formation and cell migration¹⁷.

Described herein is the identification of TMIDG1, the second family member of the IGPR-1 family. TMIGD1 (transmembrane and immunoglobulin domain containing 1) is expressed in humans, mice and other species. A recent study reported that TMIGD1 is expressed in normal human colon epithelial cells and its expression is downregulated in human colon tumors¹⁸. The data presented herein demonstrate that TMIGD1 is expressed in kidney tubular epithelial cells and that it acts to protect kidney epithelial cells from oxidative cell injury. TMIGD1 expression is downregulated in mouse AK1 and in deoxy-corticosterone acetate (DOCA) and sodium chloride (DOCA-salt)-induced chronic hypertensive kidney disease models, indicating a significant role for TMIGD1 in kidney cell injury.

### Example 2

Identification of TMIGD1 . TMIGD2 (transmembrane and Immunoglobulin domain containing 2) is a gene that encodes for a novel cell adhesion molecule “Immunoglobulin and proline rich receptor-1” (IGPR-1). The TMIGD2 gene is present in humans and some other mammals, but is not present in the mouse genome¹⁷. Further examination of the human genome revealed the presence of a TMIGD2 related gene, TMIGD1 (Ensembl gene #: ENSG00000182271, gene synonym: TMIGD, UNQ9372). TMIGD1 is located in the Chromosome 17 (Chromosome 17: 30,316,348-30,334,047) with seven putative exons encoding for a protein with 262 amino acids (FIG. 1A). The amino acid sequence of TMIGD1 is highly conserved in the humans and mice. Human TMIGD1 has greater than 90% sequence homology with mouse TMIGD1 (FIG. 1A) and the overall amino acid sequence homology of human TMIGDUIGPR-2 with human IGPR-1/TMIGD2 is about 31% (FIG. 7A). The extracellular region of TMIGD1 is predicted to contain two immunoglobulin (Ig) domains and seems to form a typical Ig fold, consisting of a sandwich of two antiparallel β-sheets (FIG. 1A). Phylogenetic tree analysis of TMIGD1 revealed that TMIGD1 gene is highly conserved among mammals and is also found in non-mammalian organisms including, Xenopus and Drosophila (FIG. 7B). One of the major differences between TMIGD1 and IGPR-1 is that TMIGD1 has a shorter cytoplasmic domain with no significant proline rich sequences (FIG. 7A). In addition, the extracellular domain of TMIGD1 contains two putative Ig domains (FIG. 1A), whereas IGPR-1 has one¹⁷.

TMIGD1 was cloned into a retroviral vector with a c-Myc tag at its C-terminus and the construct was used to express TMIGD1 in HEK-293 cells. Moreover, a polyclonal anti-TMIGD1 antibody was developed to detect TMIGD1. Analysis of cell lysates from HEK-293 cells expressing TMIGD1 showed that the anti-c-Myc antibody detects a protein band with approximate molecular weight of 45 kDa in HEK-293 cells expressing TMIGD1 (FIG. 1B). Immunoblotting with the anti-TMIGD1 antibody also detected a similar protein band in cells expressing TMIGD1 and a weaker band in the cells expressing control vector (FIG. 1B). The weaker band in HEK-293 cells expressing empty vector likely corresponds to endogenously expressed TMIGD1 in HEK-293 cells. Pre-incubation of the anti-TMIGD1 antibody with a peptide used to generate anti-TMIGD1 antibody blocked the detection of both endogenous and over-expressed TMIGD1, indicating that the anti-TMIGD1 antibody specifically recognizes TMIGD1 (FIG. 1B). The predicted molecular weight of TMIGD1 is 29 kDa, however both anti-c-Myc and anti-TMIGD1 antibodies detected TMIGD1 with an approximate molecular weight of 45 kDa. The higher molecular weight of TMIGD1 suggests that TMIGD1 is likely modified. The inventors assessed if TMIGD1 is N-glycosylated, which is common for cell surface proteins. Consistent with this possibility, treatment of cell lysate of HEK-293 cells over-expressing TMIGD1 with peptide: N-glycosidase F (PNGase F), which hydrolyzes nearly all types of N-glycans from glycoproteins, generated a 29 kDa protein which was recognized by anti-TMIGD1 antibody (FIG. 1B). The molecular weight of endogenous TMIGD1 expressed in HK2 cells was similarly reduced by PNGase F treatment (FIG. 8). The data indicates that a higher than expected molecular weight of TMIGD1 is due to its N-glycosylation.

Next, HEK-293 cells over-expressing TMIGD1 were stained with anti-c-Myc and anti-TMIGD1 antibodies. The results showed that TMIGD1 is present both at the cytoplasmic and membrane areas of HEK-293 cells (data not shown). As TMIGD1 is predicted to be a cell surface protein, a cell surface biotinylation assay was performed and it was determined that that TMIGD1 is localized at the cell surface of HEK-293 cells (FIG. 1C).

To demonstrate expression of TMIGD1 in mouse, mouse kidney tissue was assessed for expression of TMIGD1. Immunohistochemistry (IHC) analysis of mouse tissue showed that TMIGD1 is expressed in mouse kidney cells. The proximal and distal tubular epithelial cells of mouse kidney were strongly positive for TMIGD1, but TMIGD1 was very low or undetectable in podocytes (data not shown). Additionally, cell lysates derived from human kidney tubular epithelial cells (HK2) and whole cell lysate derived from human kidney were strongly positive for TMIGD1 (FIG. 9A). Analysis of additional cell lines showed that TMIGD1 is expressed in various cell lines including breast carcinoma cell lines (BT20, HCC193), lung carcinoma (H1299), bone osteosarcoma (MG63), colon carcinoma (HCT116) and normal colon epithelial cells (NCM 460), whereas its expression was low or undetectable in colon carcinoma, HT29 cells (FIG. 9B).

Given that Ig domains are known to mediate protein-protein interactions¹⁹, the inventors assessed whether the Ig domains (Ig1 and/or Ig2) of TMIGD1 mediate dimerization of TMIGD1. Using a GST-fusion of TMIGD1 encompassing two Ig domains, it was determined that TMIGD1 formed a complex with TMIGD1 (FIG. 10), demonstrating that the Ig domains of TMIGD1 act as self-dimerization domains. As TMIGD1 is predicted to act as a cell adhesion molecule, its role in mediating cell-cell interaction was assessed by subjecting cells expressing TMIGD1 to cell aggregation assays. HEK-293 cells expressing TMIGD1 formed large aggregates of cells compared to HEK-293 cells expressing an empty vector (data not shown). Also, mixing of HEK-293 cells expressing TMIGD1 with HEK-293 cells expressing the empty vector reduced formation of large cell aggregates (data not shown). The data demonstrate that over-expression of TMIGD1 in HEK-293 cells mediates cell-cell interaction. Aggregation assays are commonly used to assess the role of cell surface proteins, in particular, cell adhesion molecules in cell-cell interaction. Over-expression of proteins involved in cell-cell interaction increases cell aggregation²⁰.

### Example 3

TMIGD1 regulates trans-epithelial electric resistance and permeability: Given that TMIGD1 is similar to IGPR-1, and IGPR-1 acts as a cell adhesion molecule to regulate cell-cell adhesion¹⁷, the role of TMIGD1 in trans-epithelial electric resistance (TER) and cell permeability was examined, as cell adhesion molecules are key constituents of these cellular properties²¹. Over-expression of TMIGD1 in HEK-293 cells increased TER (FIG. 2A) and on the other hand, also reduced HEK-293 cell permeability as measured using fluorescently labeled dextran (FIG. 2B). Depleting TMIGD1 in primary human kidney epithelial cells (HK2) by siRNA significantly inhibited TER (FIG. 2C). Altogether, the data demonstrates that TMIGD1 regulates cell permeability and TER by acting as a cell adhesion molecule

Furthermore, expression of TMIGD1 in HEK-293 cells altered cell morphology and actin stress fiber formation. In HEK-293 cells expressing TMIGD1, actin stress fibrils were distinctively assembled at the periphery of cells, whereas in HEK-293 cells expressing empty vector, actin fibrils were distributed central and periphery of cells (FIG. 2D). Additionally, HEK-293 cells expressing TMIGD1 appeared to bemore spread out and were larger compared to HEK-293 cells expressing the empty vector (FIG. 2D).

Cell-cell interaction mediated by adhesion molecules such as E-cadherin and VE-cadherin inhibit cell proliferation and cell migration via contact inhibition²²⁻²⁵. To examine whether TMIGD1 regulates cell proliferation and migration, the proliferation and migration of HEK-293 cells expressing TMIGD1 was assessed. Cell proliferation was measured by the reduction of the tetrazolium dye MTT by live cells. Expression of TMIGD1 in HEK-293 cells reduced the growth rate of HEK-293 cells (FIG. 3A). Additionally, over-expression of TMIGD1 in HEK-293 cells inhibited cell migration (FIG. 3B). Given its ability to inhibit cell migration, the traction generating ability of HEK-293 cells expressing TMIGD1 was assessed using traction force microscopy. Traction force microscopy revealed that HEK-293 cells expressing TMIGD1 exhibited a significantly lower range of root mean square (RMS) traction magnitudes, with a mean value of 64.87 pa (±38.23) compared to HEK-293 cells expressing the empty vector with a mean value of 94.71 Pa (±27.89) (FIG. 3C). Taken together, the data demonstrate that TMIGD1 increases cell-cell adhesion and inhibits cell contractility and migration.

### Example 4

TMIGD1 protects human kidney epithelial cells from H₂O₂ induced cell injury and nutrient deprivation. Cell adhesion plays a major role in cell injury and repair¹⁶. Therefore, the possible function of TMIGD1 in cell injury in response to oxidative stress or nutrient deprivation was assessed. Cell viability and Annexin V apoptosis assays were used to measure the protective effect of TMIGD1 to oxidative damage induced by hydrogen peroxide. Cell viability was measured by the reduction of the tetrazolium dye, MTT. Knockdown of TMIGD1 in HK2 cells considerably reduced the survival of cells (12% versus 28%) in response to hydrogen peroxide treatment (FIG. 4A). Similarly, Annexin V staining showed that knockdown of TMIGD1 markedly increases the susceptibility of HK2 cells to hydrogen peroxide treatment (46% versus 26% cell death) (FIG. 4B). Expression of TMIGD1 in HK2 cells and the knockdown effect of TMIGD1-shRNA is shown (FIG. 4C).

Since knockdown of TMIGD1 increased cell death in response to hydrogen peroxide, the ability of over-expression of TMIGD1 to protect cells from hydrogen peroxide-induced cell injury was assessed. Over-expression of TMIGD1 in HEK293 cells increased survival of cells in response to hydrogen peroxide treatment, as measured by cell viability (FIG. 4D) and Annexin V staining (FIG. 4E). Additionally, knockdown of TMIGD1 decreased survival of HK2 cells in nutrient poor medium (serum free, 5 mM glucose), whereas over-expression of TMIGD1 in HEK-293 cells increased survival (FIGS. 11A-11B). Taken together, the data demonstrate that TMIGD1 regulates survival of epithelial cells in response to cell injury.

Next, the regulation of expression of TMIGD1 by oxidative stress was assessed. Treatment of HK2 cells with hydrogen peroxide downregulated TMIGD1 (FIG. 5A) and treatment of cells with a proteosome inhibitor, Bortezomib, rescued downregulation of TMIGD1 in response to hydrogen peroxide treatment (FIG. 5B). The data demonstrate that hydrogen peroxide-induced cell injury downregulates TMIGD1. Because protein degradation is often mediated by ubiquitination which targets proteins for lysosomal and proteasomal degradation²⁶, the ubiquitination of TMIGD1 in HK2 cells in response to hydrogen peroxide was assessed. The treatment of HK2 cells with hydrogen peroxide was determined to induce ubiquitination of TMIGD1 (FIG. 5C). The data demonstrate that hydrogen peroxide downregulates TMIGD1 by promoting its ubiquitination.

Next, it was assessed if preventing the downregulation of TMIGD1 in response to hydrogen peroxide by Bortezomib, could improve the survival of cells from hydrogen peroxide-induced cell injury. The result demonstrate that co-treatment of HK2 cells with hydrogen peroxide and Bortezomib reverses cell death caused by hydrogen peroxide (FIG. 5D). Taken together, the data demonstrate that over-expression of TMIGD1 plays a protective role and promotes cell survival under nutrient or oxygen deprived conditions. Furthermore, cell-damaging oxidative agents downregulate expression and promote ubiquitination of TMIGD1.

Expression of TMIGD1 is reduced during kidney cell injury. Since TMIGD1 plays a protective role in cell injury, and oxidative stress downregulates TMIGD1, the expression status of TMIGD1 in chronic kidney disease (CKD) was assessed in a hypertensive mouse model, and an acute renal ischemia reperfusion (IR) model. Tubular epithelial cell injury and cell death due to apoptosis and necrosis represent the common denominator of both models^{1,27,28}. Upon IR, tubular injury was observed in kidneys, which underwent IR after 48 hours compared to kidneys that did not receive the treatment. There was marked epithelial cell necrosis involving most of the corticomedullary tubules (FIG. 6A).

Immunohistochemistry analysis of normal and ischemic perfused kidney sections after ischemia showed that after IR for 48 hours, there was a marked reduction in the staining of TMIGD1 in many epithelial cells lining the proximal tubules as compared to staining observed in the untreated samples (data not shown). The reduced staining of TMIGD1 was prominent mainly in the S3 segment in the outer medullary rays corresponding to site of injury that is mainly affected in line with the kind of injury initiated.

Moreover, the expression of TMIGD1 was also assessed in another mouse kidney disease model, namely in the DOCA salt-induced hypertensive mouse model of kidney. The result demonstrated that expression of TMIGD1 was substantially reduced in the morphologically normal tubules compared to tubules in the non-hypertensive mice after 24 hours and 2 weeks of inducing hypertension (FIG. 6B). Taken together, the data demonstrate that expression of TMIGD1 is markedly reduced in the experimental mouse models of ischemia reperfusion (IR) injury and CKD. Furthermore, these findings suggest that TMIGD1 expression is associated with survival of kidney tubules during kidney injury.

### Example 5

The inventors have discovered herein that TMIGD1 is a novel adhesion molecule expressed in kidney tubular epithelial cells, and promotes the survival of kidney epithelial cells from oxidative cell injury. Kidneys are composed of hundreds of thousand (or more) of functionally independent units called nephrons, and each nephron independently controls its own permeability and solute transport capabilities by changing its adhesive characteristics based on external signals. The normal function of nephrons is highly dependent on epithelial cell-cell adhesion and the surrounding extracellular matrix^{16,29} and adhesion molecules such as cadherins and integrins are key mediators of adhesion^{30,31}. The inventors have discovered herein that TMIGD1 regulates kidney epithelial cell permeability, as demonstrated by the discovery that over-expression of TMIGD1 increased trans-epithelial electric resistance (TER) and reduced permeability; and that reducing expression of TMIGD1 abrogated TER. Consistent with the classical function of cell adhesion molecules, over-expression of TMIGD1 in HEK-293 cells reduced cell migration, cell proliferation, and altered the actin fibril assembly, indicating that TMIGD1 expression in epithelial cells plays a functional role in epithelial cell function.

More importantly, the data presented herein demonstrates that TMIGD1 is involved in ischemic renal cell injury. Reducing expression of TMIGD1 in HK2 cells significantly reduced survival of cells in response to hydrogen peroxide-induced cell injury. Similarly, over-expression of TMIGD1 in HEK-293 cells increased cell survival in response to hydrogen peroxide-mediated cell injury. Given that loss of adhesion in renal ischemia is linked to cell death^{9,32}, the inventors have discovered that TMIGD1 plays significant role in renal cell injury, and can be used to treat kidney injury and/or kidney cancer. Curiously, the recent study reports that TMIGD1 expression is downregulated in human colon tumors¹⁸, indicating that in colon tumors the loss of TMIGD1 contributes to increases in tumor cells migration and proliferation, consistent with this possibility TMIGD1 over-expression in HEK-293 cells inhibited both cell proliferation and migration (FIG. 3A-3C).

Underscoring the functional importance of TMIGD1 in ischemia-induced cell injury, hydrogen peroxide treatment of HK2 cells resulted in the ubiquitination and degradation of TMIGD1. Blocking the ubiquitination pathway by adding Bortezomib reversed downregulation of TMIGD1 by hydrogen peroxide. Interestingly, Bortezomib treatment of HK2 cells inhibited cell death in response to hydrogen peroxide. Recently, Bortezomib treatment has been studied as a possible therapeutic intervention in acute renal failure³³. The data disclosed herein demonstrate that agents that up-regulate expression of, or activate the function of TMIGD1 are a new class of drugs for treatment of renal failure. In support of a possible role of TMIGD1 in renal injury in vivo, TMIGD1 expression was detected to be was significantly downregulated in mouse models of renal injury. Loss of TMIGD1 may contribute in part to loss of cell adhesion and death in kidney epithelial cells.

Despite recent progress in renal therapy and critical care medicine, acute kidney injury still carries a high mortality rate¹, underscoring a need for a better understating of the biology of renal cell injury and the molecules involved. Identification of TMIGD1 as a putative molecule involved in renal cell injury and further characterization of its role and the mechanisms involved, should shed new light on molecular mechanism of renal cell injury.

### Example 6

Identification of TMIGD1 as a Novel Candidate Tumor Suppressor Gene

The majority of human kidney cancers start as benign adenomas through loss of the von Hippel-Lindau (VHL) tumor suppressor gene. This early genetic lesion often progress to invasive carcinomas through additional changes, the mechanism of which is poorly understood. Renal cell carcinoma (RCC) is common and highly metastatic urological cancer with poor prognosis. As described above herein, transmembrane and immunoglobulin domain-containing 1 (TMIGD1) is a novel immunoglobulin (Ig) domain containing adhesion molecule (Ig-CAM) highly expressed in renal tubular epithelial cells. TMIGD1 is predominantly expressed in human renal epithelial cells and its expression is significantly downregulated in human RCC. Reestablishing expression TMIGD1 in renal tumor cell line, 786-0 decreased tumor growth and cell migration in cell culture, the key tumorigenic characteristics of cancerous cells.

Furthermore, TMIGD1 downregulation in RCC is linked to loss of pVHL, as re-expressing VHL in RCC tumor cell lines partially restored expression of TMIGD1. Furthermore, identified herein is Cysteine-rich angiogenic inducer 61 (CYR61) (also referred to in the art as CCN family member 1 (CCN1), IGFBP10, GIG1), as a putative ligand for TMIGD1. Taken together, the data demonstrate that TMIGD1 is a novel candidate tumor suppressor gene and its expression status can be exploited as a putative RCC biomarker. Furthermore, therapeutic strategies aiming to restore expression of TMIGD1 in RCC can permit an anti-cancer therapy.

Renal cell carcinoma (RCC), the most common urological cancer, is composed of a heterogeneous group of kidney epithelial tumors with variable genetic and clinical outcomes. RCC is a high-risk metastasizing tumor that has a poor prognosis as there are very few tumor markers for renal cancer (Linehan, W. M. et al. 2010) and it is generally insensitive to conventional chemo and radiotherapies (Walsh, N. et al. 2009) or targeted therapeutics, including anti-angiogenesis drugs and mTOR inhibitors (Pirrotta, M. T. et al. (2011). Clear cell renal cell carcinoma (CCRCC) represents the most common RCC subtype, which makes up about 70% of all cancers of kidney and is commonly associated with mutation or loss of the von Hippel-Lindau (VHL) tumor suppressor gene. Papillary and chromophobe renal cell carcinomas represent the second and the third most common RCC tumors.

Although loss of pVHL function is associated with RCC development, the molecular mechanism by which loss of pVHL contributes to RCC development and progression is not fully understood. The aim of the present study was to investigate expression and functional importance of transmembrane and immunoglobulin domain-containing 1 (TMIGD1) in RCC. TMIGD1 is a immunoglobulin (Ig) domain containing adhesion molecule (Ig-CAM), which is highly expressed in renal tubular epithelial cells and acts to protect epithelial cells from oxidative cell injury (Arfa et al., 2015). TMIGD1 expression in renal epithelial cells is significantly altered in both acute kidney injury and in deoxy-corticosterone acetate and sodium chloride (deoxy-corticosterone acetate salt)-induced chronic hypertensive kidney disease mouse models (Arfa et al., 2015).

As disclosed herein, TMIGD1 expression is downregulated in human renal cancer. The expression of TMIGD1 was examined in a panel of human organs/tissues consisting of ovary, heart, vein, kidney, lung, liver, brain, pancreas, bone marrow and skin using quantitative PCR (qPCR). The results demonstrated that TMIGD1 expression was highest in kidney (FIG. 12A). Expression of TMIGD1 was very low, or undetectable, in ovary, heart, vein, lung, liver, pancreas, bone marrow and skin, except brain. TMIGD1 was expressed in brain, albeit at the significantly lower level compared to its expression in kidney (FIG. 12A). Furthermore, expression of TMIGD1 at the protein level was also highest in kidney followed by brain as determined by western blot analysis (FIG. 12B). TMIGD1 protein was not detected in lung, liver, heart and skin (FIG. 12B). Consistent with qPCR and western blot analysis, immunohistochemistry (IHC) analysis of human kidney tissue also showed that TMIGD lwas highly expressed in human kidney (data not shown). Kidney tubular epithelial cells were particularly positive for TMIGD1, whereas podocytes of the glomerulus were negative (data not shown).

Expression of TMIGD1 was also assessed in a cohort of 20 formalin fixed human renal cancer biopsy samples by IHC analysis. The cohort consisted of 9 renal cell carcinomas (4 clear cell, 3 chromophobe, and 2 papillary renal cell carcinomas), and 11 cases of benign kidney lesions or normal kidney tissues. IHC analysis demonstrated that TMIGD1 was significantly downregulated in RCC cancers (9 cases) compared to normal adjacent tissue or other normal/benign kidney (11 cases) (FIG. 13). Interestingly, expression of TMIGD1 in all three distinct histological RCC tumor types (clear cell, chromophobe and papillary renal cell carcinomas) was downregulated (FIG. 13).

To examine expression of TMIGD1 further in RCC, expression of TMIGD1 was analyzed in a panel of RCC tumor cell lines by qPCR and western blot analysis. The qPCR analysis showed that at the mRNA level, TMIGD1 is expressed relatively at low level in human RCC tumor cell lines including, TK10, 7860, A498 and 769P compared to its expression in normal kidney (data not shown, also see FIGS. 15A-15C). However, TMIGD1 at the protein level, as determined by western blot analysis was hardly detectable in RCC tumor cell lines (data not shown, FIGS. 15A-15C). Taken together, the data indicate that TMIGD1 is highly expressed in normal human kidney epithelial cells, but its expression is significantly downregulated in human RCC and RCC tumor cell lines.

Reconstitution of TMIGD1 expression in RCC tumor cells inhibits cell proliferation and migration. Next, the effect of re-expression of TMIGD1 in RCC cell line, 786-0 was analyzed. MTT and BrdU assays were used to measure the effect of TMIGD1 in proliferation of 786-O cells. The result showed that re-expression of TMIGD1 in 786-O cells inhibits tumor cell proliferation (FIG. 14A-14B). Furthermore, re-expression of TMIGD1 in 786-O cells also inhibited tumor cell migration (FIG. 14C). Taken together, the data indicate that loss of TMIGD1 in tumor cells is linked to tumorigenic and invasive function of RCC tumors.

TMIGD1 expression in RCC is regulated by VHL. Given that the loss of pVHL is commonly associated with RCC, it was hypothesized that the loss of pVHL function in RCC tumor cells is linked to pVHL. To this end, qPCR was used to examine expression of TMIGD1 in a panel of RCC tumors. The result showed that TMIGD1 is highly expressed in pVHL positive tumor cell lines, CAKi-1 and TK10 cells but significantly low in pVHL negative cells, A498, UMRC6 and 769P (FIG. 15A). Further western blot analysis showed that TMIGD1 expression at the protein level in CAKi-1 and TK10 but not in A498, UMRC6 and 769P cells (FIG. 15A). To examine whether the loss of pVHL in RCC tumor cell lines is associated with loss of pVHL, functional pVHL was over-expressed in two pVHL negative cell lines, 786-O and A498 cells and expression of TMIGD1 was examined. The result showed that re-expression of functional pVHL in 786-O and A498 cells significantly increases expression of TMIGD1 both at the mRNA and protein levels (FIG. 15C).

Identification of CYR61 as a putative ligand for TMIGD1. To identify potential ligand for TMIGD1, Glutathione S-transferase (GST)-fusion proteins encompassing the extracellular domain of TMIGD1 were generated and used to identify potential ligands for TMIGD1. To this end, the purified recombinant GST-TMIGD1 fusion protein attached to glutathione beads were incubated with cell lysates of human colon tumor cell line, RKO and GST-TMIGD1-associated proteins were resolved on SDS-PAGE gel. Protein bands were visualized by coomassie blue and protein bands were excised and subjected mass spectrometry analysis. Cysteine-rich angiogenic inducer 61 (CYR61) also called CCN family member 1 (CCN1) was identified among a few other proteins as a TMIGD1 binding protein (FIG. 16A). Further in vitro binding assay validated the binding of CYR61 to TMIGD1 (FIGS. 16B & 16C).

Cyr61 is a soluble secreted protein that promotes diverse biological responses in a context dependent manner. In endothelial cells it mediates cell adhesion, migration and proliferation formation through binding to integrin alpha-6/beta-1, integrin alpha-v/beta-5 and integrin alpha-v/beta-3. Cyr61 also exhibits both tumorigenic and tumor suppressor activities depending tumor types. For example, in breast, renal, gastric, squamous cell, and colorectal carcinomas it stimulates tumor growth. However, in other tumor types such as small cell lung cancer (Tong et al., 2001), endometrial cancer (Wenwen Chien, et al., 2004), human hepatocellular carcinoma (Feng et al., 2008) and melanoma (Dobroff et al., 2009) acts as a tumor suppressor. To date, the molecular mechanism responsible for contradictory function of CYR61 as a both tumorigenic and tumor suppressor in human cancers is not known. Identification of CYR61 as a putative ligand for TMIGD1 indicate that in certain conditions such as oxidative stress-induced cell injury, CYR61 binding to TMIGD1 could regulate the anti-proliferative and pro-survival activity of TMIGD1.

### REFERENCES

The references disclosed herein and throughout the specification are incorporated herein in their entirety.
1. Schrier R W, Wang W, Poole B, Mitra A: Acute renal failure: definitions, diagnosis, pathogenesis, and therapy, The Journal of clinical investigation 2004, 114:5-14
2. Okusa M D, Chertow G M, Portilla D, Acute Kidney Injury Advisory Group of the American Society of N: The nexus of acute kidney injury, chronic kidney disease, and World Kidney Day 2009, Clinical journal of the American Society of Nephrology: CJASN 2009, 4:520-522
3. Xue J L, Daniels F, Star R A, Kimmel P L, Eggers P W, Molitoris B A, Himmelfarb J, Collins A J: Incidence and mortality of acute renal failure in Medicare beneficiaries, 1992 to 2001, Journal of the American Society of Nephrology: JASN 2006, 17:1135-1142
4. Chawla L S, Eggers P W, Star R A, Kimmel P L: Acute kidney injury and chronic kidney disease as interconnected syndromes, The New England journal of medicine 2014, 371:58-66
5. Grgic I, Campanholle G, Bijol V, Wang C, Sabbisetti V S, Ichimura T, Humphreys B D, Bonventre J V: Targeted proximal tubule injury triggers interstitial fibrosis and glomerulosclerosis, Kidney international 2012, 82:172-183
6. Sanz A B, Santamaria B, Ruiz-Ortega M, Egido J, Ortiz A: Mechanisms of renal apoptosis in health and disease, Journal of the American Society of Nephrology: JASN 2008, 19:1634-1642
7. Versteilen A M, Di Maggio F, Leemreis J R, Groeneveld A B, Musters R J, Sipkema P: Molecular mechanisms of acute renal failure following ischemia/reperfusion, The International journal of artificial organs 2004, 27:1019-1029
8. Myrvang H: Acute kidney injury: Obesity is associated with AKI after surgery via oxidative stress, Nature reviews Nephrology 2012, 8:433
9. Sutton T A, Molitoris B A: Mechanisms of cellular injury in ischemic acute renal failure, Seminars in nephrology 1998, 18:490-497
10. Molitoris B A: Actin cytoskeleton in ischemic acute renal failure, Kidney international 2004, 66:871-883
11. Denker B M, Nigam S K: Molecular structure and assembly of the tight junction, The American journal of physiology 1998, 274:F1-9
12. Thadhani R, Pascual M, Bonventre J V: Acute renal failure, The New England journal of medicine 1996, 334:1448-1460
13. Heyman S N, Lieberthal W, Rogiers P, Bonventre J V: Animal models of acute tubular necrosis, Current opinion in critical care 2002, 8:526-534
14. Alejandro V S, Strafuss A C: Microscopic postmortem changes in kidneys of the domestic fowl, Avian diseases 1984, 28:586-607
15. Duffield J S, Park K M, Hsiao L L, Kelley V R, Scadden D T, Ichimura T, Bonventre J V: Restoration of tubular epithelial cells during repair of the postischemic kidney occurs independently of bone marrow-derived stein cells, The Journal of clinical investigation 2005, 115:1743-1755
16. Prozialeck W C, Edwards J R: Cell adhesion molecules in chemically-induced renal injury, Pharmacology & therapeutics 2007, 114:74-93
17. Rahimi N, Rezazadeh K, Mahoney J E, Hartsough E, Meyer R D: Identification of IGPR-1 as a novel adhesion molecule involved in angiogenesis, Molecular biology of the cell 2012, 23:1646-1656
18. Cattaneo E, Laczko E, Buffoli F, Zorzi F, Bianco M A, Menigatti M, Bartosova Z, Haider R, Helmchen B, Sabates-Bellver J, Tiwari A, Jiricny J, Marra G: Preinvasive colorectal lesion transcriptomes correlate with endoscopic morphology (polypoid vs. nonpolypoid), EMBO molecular medicine 2011, 3:334-347
19. Barclay A N: Membrane proteins with immunoglobulin-like domains—a master superfamily of interaction molecules, Seminars in immunology 2003, 15:215-223
20. Balzar M, Briaire-de Bruijn I H, Rees-Bakker H A, Prins F A, Helfrich W, de Leij L, Riethmuller G, Alberti S, Warnaar S O, Fleuren G J, Litvinov S V: Epidermal growth factor-like repeats mediate lateral and reciprocal interactions of Ep-CAM molecules in homophilic adhesions, Molecular and cellular biology 2001, 21:2570-2580
21. Anderson J M, Van Itallie C M: Physiology and function of the tight junction, Cold Spring Harbor perspectives in biology 2009, 1:a002584
22. Stockinger A, Eger A, Wolf J, Beug H, Foisner R: E-cadherin regulates cell growth by modulating proliferation-dependent beta-catenin transcriptional activity, The Journal of cell biology 2001, 154:1185-1196
23. Castilla M A, Arroyo M V, Aceituno E, Aragoncillo P, Gonzalez-Pacheco F R, Texeiro E, Bragado R, Caramelo C: Disruption of cadherin-related junctions triggers autocrine expression of vascular endothelial growth factor in bovine aortic endothelial cells: effects on cell proliferation and death resistance, Circulation research 1999, 85:1132-1138
24. Lauffenburger D A, Schaffer D V: The matrix delivers, Nature medicine 1999, 5:733-734
25. Ryan P L, Foty R A, Kohn J, Steinberg M S: Tissue spreading on implantable substrates is a competitive outcome of cell-cell vs. cell-substratum adhesivity, Proceedings of the National Academy of Sciences of the United States of America 2001, 98:4323-4327
26. Rahimi N: The ubiquitin-proteasome system meets angiogenesis, Molecular cancer therapeutics 2012, 11:538-548
27. Devarajan P: Update on mechanisms of ischemic acute kidney injury, Journal of the American Society of Nephrology: JASN 2006, 17:1503-1520
28. Chen D, Coffman T M: The kidney and hypertension: lessons from mouse models, The Canadian journal of cardiology 2012, 28:305-310
29. Schneeberger E E, Lynch R D: The tight junction: a multifunctional complex, American journal of physiology Cell physiology 2004, 286:C1213-1228
30. Schwartz M A, Ginsberg M H: Networks and crosstalk: integrin signalling spreads, Nature cell biology 2002, 4:E65-68
31. Novak A, Hsu S C, Leung-Hagesteijn C, Radeva G, PapkoffJ, Montesano R, Roskelley C, Grosschedl R, Dedhar S: Cell adhesion and the integrin-linked kinase regulate the LEF-1 and beta-catenin signaling pathways, Proceedings of the National Academy of Sciences of the United States of America 1998, 95:4374-4379
32. van Wetering S, van Buul J D, Quik S, Mul F P, Anthony E C, ten Klooster J P, Collard J G, Hordijk P L: Reactive oxygen species mediate Rac-induced loss of cell-cell adhesion in primary human endothelial cells, Journal of cell science 2002, 115:1837-1846
33. Ludwig H, Drach J, Graf H, Lang A, Meran J G: Reversal of acute renal failure by bortezomib-based chemotherapy in patients with multiple myeloma, Haematologica 2007, 92:1411-1414
34. Rahimi N, Dayanir V, Lashkari K: Receptor chimeras indicate that the vascular endothelial growth factor receptor-1 (VEGFR-1) modulates mitogenic activity of VEGFR-2 in endothelial cells, The Journal of biological chemistry 2000, 275:16986-16992
35. Srinivasan S, Meyer R D, Lugo R, Rahimi N: Identification of PDCL3 as a novel chaperone protein involved in the generation of functional VEGF receptor 2, The Journal of biological chemistry 2013, 288:23171-23181
36. Matter K, Balda M S: Functional analysis of tight junctions, Methods 2003, 30:228-234
37. Meyer R D, Srinivasan S, Singh A J, Mahoney J E, Gharahassanlou K R, Rahimi N: PEST motif serine and tyrosine phosphorylation controls vascular endothelial growth factor receptor 2 stability and downregulation, Molecular and cellular biology 2011, 31:2010-2025
38. Singh A J, Meyer R D, Navruzbekov G, Shelke R, Duan L, Band H, Leeman S E, Rahimi N: A critical role for the E3-ligase activity of c-Cbl in VEGFR-2-mediated PLCgamma1 activation and angiogenesis, Proceedings of the National Academy of Sciences of the United States of America 2007, 104:5413-5418
39. Meyer R D, Singh A J, Rahimi N: The carboxyl terminus controls ligand-dependent activation of VEGFR-2 and its signaling, The Journal of biological chemistry 2004, 279:735-742
40. Butler J P, Tolic-Norrelykke I M, Fabry B, Fredberg J J: Traction fields, moments, and strain energy that cells exert on their surroundings, American journal of physiology Cell physiology 2002, 282:C595-605
41. Wang Z, Gall J M, Bonegio R G, Havasi A, Hunt C R, Sherman M Y, Schwartz J H, Borkan S C: Induction of heat shock protein 70 inhibits ischemic renal injury, Kidney international 2011, 79:861-870
Ringo et al., Extracellular Matrix protein CCN1 (CYR61) negatively regulated endothelial cell adhesion molecules. PhD Thesis, University of Rochester. 2015

SEQUENCES:

```
SEQ ID NO: 9 (Human CYR61 amino acid sequence, NP_001545.2)
MSSRIARALALVVTLLHLTRLALSTCPAACHCPLEAPKCAPGVG
LVRDGCGCCKVCAKQLNEDCSKTQPCDHTKGLECNFGASSTALKGICRAQSEGRPCEY
NSRIYQNGESFQPNCKHQCTCIDGAVGCIPLCPQELSLPNLGCPNPRLVKVTGQCCEE
WVCDEDSIKDPMEDQDGLLGKELGFDASEVELTRNNELIAVGKGSSLKRLPVEGMEPR
ILYNPLQGQKCIVQTTSWSQCSKTCGTGISTRVTNDNPECRLVKETRICEVRPCGQPV
YSSLKKGKKCSKTKKSPEPVRFTYAGCLSVKKYRPKYCGSCVDGRCCTPQLTRTVKMR
FRCEDGETFSKNVMMIQSCKCNYNCPHANEAAFPFYRLFNDIHKFRD
SEQ ID NO: 10 (human TMIGD1 amino acid sequence)
      1 mawkssvimg mgrflllvil flpremtssv ltvngkteny ildttpgsqa slicavqnht
    61 reeellwyre egrvdlksgn kinsssvcvs sisendngis ftcrlgrdqs vsvsvvlnvt
  121 fppllsgndf qtveegsnvk lvcnvkanpq aqmmwyknss lldleksrhq iqqtsesfql
  181 sitkvekpdn gtydciakss lktesldfhl ivkdktvgvp iepiiaacvv ifltlcfgli
  241 arrkkimklc mkdkdphset al
SEQ ID NO: 11 (human TMIGD1-alternatively spliced variant)
      1 mawkssvimg mgrflllvil flpremtssv ltvngkteny ildttpgsqa slicavqnht
    61 reeellwyre egrvdlksgn kinsssvcvs sisendngis ftcrlgrdqs vsvsvvlnvt
  121 fppllsgndf qtveegsnvk lvcnvkanpq aqmmwyknss lldleksrhq iqqtsesfql
  181 sitkvekpdn gtydciakss lktesldfhl ivkalheg
SEQ ID NO: 12 (Ig1 of human TMIGD1, corresponding to amino acids
44-123 of SEQ ID NO: 10)
TTPGSQA SLICAVQNHT REEELLWYRE EGRVDLKSGN KINSSSVCVS SISENDNGIS
FTCPLGRDQS VSVSVVLNVT FPP
SEQ ID NO: 13 (Ig1 of human TMIGD1, corresponding to amino acids
124-215 of SEQ ID NO: 10)
Ig2: LLSGNDF QTVEEGSNVK LVCNVKANPQ AQMMWYKNSS LLDLEKSPHQ IQQTSESPQL
  181 SITKVEKPDN GTYSCIAKSS LKTESLDFHL IVKDK
SEQ ID NO: 14: mRNA encoding human CYR61 (NM_001554.4)
      1 agaccgcgag cgagagcgcc cccgagcagc gcccgcgccc tccgcgcctt ctccgccggg
    61 acctcgagcg aaagacgccc gcccgccgcc cagccctcgc ctccctgccc accgggccca
  121 ccgcgccgcc accccgaccc cgctgcgcac ggcctgtccg ctgcacacca gcttgttggc
  181 gtcttcgtcg ccgcgctcgc cccgggctac tcctgcgcgc cacaatgagc tcccgcatcg
  241 ccagggcgct cgccttagtc gtcacccttc tccacttgac caggctggcg ctctccacct
  301 gccccgctgc ctgccactgc cccctggagg cgcccaagtg cgcgccggga gtcgggctgg
  361 tccgggacgg ctgcggctgc tgtaaggtct gcgccaagca gctcaacgag gactgcagca
  421 aaacgcagcc ctgcgaccac accaaggggc tggaatgcaa cttcggcgcc agctccaccg
  481 ctctgaaggg gatctgcaga gctcagtcag agggcagacc ctgtgaatat aactccagaa
  541 tctaccaaaa cggggaaagt ttccagccca actgtaaaca tcagtgcaca tgtattgatg
  601 gcgccgtggg ctgcattcct ctgtgtcccc aagaactatc totccocaac ttgggctgtc
  661 ccaaccctcg gctggtcaaa gttaccgggc agtgctgcga ggagtgggtc tgtgacgagg
  721 atagtatcaa ggaccccatg gaggaccagg acggcctcct tggcaaggag ctgggattcg
  781 atgcctccga ggtggagttg acgagaaaca atgaattgat tgcagttgga aaaggcagct
  841 cactgaagcg gctccctgtt tttggaatgg agcctcgcat cctatacaac cctttacaag
  901 gccagaaatg tattgttcaa acaacttcat ggtcccagtg ctcaaagacc tgtggaactg
  961 gtatctccac acgagttacc aatgacaacc ctgagtgccg ccttgtgaaa gaaacccgga
1021 tttgtgaggt gcggccttgt ggacagccag tgtacagcag cctgaaaaag ggcaagaaat
1081 gcagcaagac caagaaatcc cccgaaccag tcaggtttac ttacgctgga tgtttgagtg
1141 tgaagaaata ccggcccaag tactgcggtt cctgcgtgga cggccgatgc tgcacgcccc
1201 agctgaccag gactgtgaag atgcggttcc gctgcgaaga tggggagaca ttttccaaga
1261 acgtcatgat gatccagtcc tgcaaatgca actacaactg cccgcatgcc aatgaagcag
1321 cgtttccctt ctacaggctg ttcaatgaca ttcacaaatt tagggactaa atgctacctg
1381 ggtttccagg gcacacctag acaaacaagg gagaagagtg tcagaatcag aatcatggag
1441 aaaatgggcg ggggtggtgt gggtgatggg actcattgta gaaaggaagc cttgctcatt
1501 cttgaggagc attaaggtat ttcgaaactg ccaagggtgc tggtgcggat ggacactaat
1561 gcagccacga ttggagaata ctttgcttca tagtattgga gcacatgtta ctgcttcatt
1621 ttggagcttg tggagttgat gactttctgt tttctgtttg taaattattt gctaagcata
1681 ttttctctag gcttttttcc ttttggggtt ctacagtcgt aaaagagata ataagattag
1741 ttggacagtt taaagctttt attcgtcctt tgacaaaagt aaatgggagg gcattccatc
1801 ccttcctgaa gggggacact ccatgagtgt ctgtgagagg cagctatctg cactctaaac
1861 tgcaaacaga aatcaggtgt tttaagactg aatgttttat ttatcaaaat gtagcttttg
1921 gggagggagg ggaaatgtaa tactggaata atttgtaaat gattttaatt ttatattcag
1981 tgaaaagatt ttatttatgg aattaaccat ttaataaaga aatatttacc taatatctga
2041 gtgtatgcca ttcggtattt ttagaggtgc tccaaagtca ttaggaacaa cctagctcac
2101 gtactcaatt attcaaacag gacttattgg gatacagcag tgaattaagc tattaaaata
2161 agataatgat tgcttttata ccttcagtag agaaaagtct ttgcatataa agtaatgttt
2221 aaaaaacatg tattgaacac gacattgtat gaagcacaat aaagattctg aagctaaatt
2281 tgtgatttaa gaaaa
```

1. A method of treating kidney disease or cancer in a subject in need thereof, the method comprising administering a TMIGD1 agonist to the subject.

2. The method of claim 1, wherein the agonist is selected from the group consisting of:
an antibody reagent; a TMIGD1 polypeptide; and a nucleic acid encoding a TMIGD1 polypeptide.

3. The method of claim 1, wherein the agonist is a polypeptide comprising the sequence of SEQ ID NO: 9 (CYR61) or a polypeptide having at least 80% sequence identity to SEQ ID NO: 9.

4. The method of claim 1, wherein the kidney cancer is renal cell carcinoma (RCC).

5. The method of claim 1, wherein the kidney disease is selected from the group consisting of:
acute kidney injury (AKI); chronic kidney disease (CKD); kidney cysts; Alport Syndrome; Diabetic Nephropathy; Fabry Disease; Focal Segmental Glomerulosclerosis; Glomerulonephritis; IgA Nephropathy (Berger's Disease); Kidney Stones; and Polycystic Kidney Disease (PKD).

6. The method of claim 2, wherein the antibody reagent is an antibody or antigen binding fragment thereof.

7. The method of claim 6, wherein the antibody is selected from the group consisting of polyclonal antibody, monoclonal antibody, humanized or chimeric antibody or human monoclonal antibody.

8. The method of claim 1, wherein the antibody reagent specifically binds to the extracellular domain of the TMIGD1 polypeptide corresponding to amino acids 41-215 of SEQ ID NO: 10.

9. The method of claim 1, wherein the antibody reagent specifically binds to any region of: Ig1 (corresponding to amino acids 44-123 of SEQ NO: 10), Ig2 (corresponding to amino acids 124-215 of SEQ ID NO: 10) or Ig1 and Ig2.

10. The method of claim 1, wherein the antibody reagent blocks the interaction or binding of CYR61 with the extracellular domain of the TMIGD1 polypeptide corresponding to amino acids 41-215 of SEQ ID NO: 10.

11. An isolated activating antibody which specifically binds the CYR61-binding domain of TMIGD1, thereby activating TMIGD1.

12. The antibody of claim 11, wherein the CYR61 binding domain is located in the extracellular domain of the TMIGD1 polypeptide corresponding to amino acids 41-215 of SEQ ID NO: 10.

13. The antibody of claim 11, wherein the activating antibody specifically binds to at least 1 or more amino acids in any region of: Ig1 (corresponding to amino acids 44-123 of SEQ ID NO: 10), Ig2 (corresponding to amino acids 124-215 of SEQ ID NO: 10) or Ig1 and Ig2.

14. The antibody of claim 11, wherein the activating antibody competes with CYR61 for specific binding to TMIGD1.

15. The antibody of claim 11, wherein the activating antibody is selected from: a polyclonal antibody, a monoclonal antibody, a human antibody, a humanized antibody a chimeric antibody or antigen-binding fragments thereof.

16. A TMIGD1 agonist comprising a nucleic acid sequence encoding a TMIGD1 polypeptide.

17. (canceled)

18. (canceled)

19. (canceled)

20. A TMIGD1 agonist comprising a polypeptide comprising the amino acid sequence of SEQ ID NO: 9 (CYR61) or having an amino acid sequence of at least 80% sequence identity to SEQ ID NO: 9.

21. (canceled)

22. A method of treating kidney cancer in a subject in need thereof, the method comprising administering a treatment for kidney cancer to a subject determined to have a decreased level of TMIGD1.

23. The method of claim 22, further comprising a first step of determining the level of TMIGD1 in the subject.

24. (canceled)

25. (canceled)

26. (canceled)

27. (canceled)

28. (canceled)


SEQUENCE LISTING (33 sequences)

SEQ ID NO: 1 - protein, 262 aa, Homo sapiens
MAWKSSVIMQMGRFLLLVILFLPREMTSSVLTVNGKTENYILDTTPGSQASLICAVQNHTREEELLWYREEGRVDLKSGNKINSSSVCVSSISENDNGISFTCRLGRDQSVSVSVVLNVTFPPLLSGNDFQTVEEGSNVKLVCNVKANPQAQMMWYKNSSLLDLEKSRHQIQQTSESFQLSITKVEKPDNGTYSCIAKSSLKTESLDFHLIVKDKTVGVPIEPIIAACVVIFLTLCFGLIARRKKIMKLCMKDKDPHSETAL

SEQ ID NO: 2 - protein, 261 aa, Mus sp.
MVWKITGPLQACQLLLVVLSLPQGRTSSVLTVNGRTENYILDTQHGVQASLECAVQNHTEDEELLWYREDGIVDLKNGNKINISSVCVSPINESDNGVRFTCKLQRDQTVSVTVVLNVTFPPLLSGNGFQTVEENSDVSLVCNVKSNPQAQMMWYKNNSALVLEKGRHQIHQTRESFQLSITKVKKSDNGTYSCIASSSLKMETMDFHLLVKDKVFVMPAEPIIAACVVVVLTMAFALFSRRKRIMKLCGKKNDPNSETAL

SEQ ID NO: 3 - protein, 282 aa, Homo sapiens
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGPPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 4 - protein, 261 aa, Mus sp.; identical to SEQ ID NO: 2

SEQ ID NO: 5 - DNA, 19 bp, Artificial Sequence
  Description of Artificial Sequence Synthetic primer
gacccgaattcagaaacac

SEQ ID NO: 6 - DNA, 17 bp, Artificial Sequence
  Description of Artificial Sequence Synthetic primer
gcccttctcaaaacgta

SEQ ID NO: 7 - DNA, 20 bp, Artificial Sequence
  Description of Artificial Sequence Synthetic primer
cgccgctagaggtgaaattc

SEQ ID NO: 8 - DNA, 20 bp, Artificial Sequence
  Description of Artificial Sequence Synthetic primer
ggtatttgctacggctgacc

SEQ ID NO: 9 - protein, 381 aa, Homo sapiens [sequence omitted]

SEQ ID NO: 10 - protein, 262 aa, Homo sapiens; identical to SEQ ID NO: 1

SEQ ID NO: 11 - protein, 218 aa, Homo sapiens
MAWKSSVIMQMGRFLLLVILFLPREMTSSVLTVNGKTENYILDTTPGSQASLICAVQNHTREEELLWYREEGRVDLKSGNKINSSSVCVSSISENDNGISFTCRLGRDQSVSVSVVLNVTFPPLLSGNDFQTVEEGSNVKLVCNVKANPQAQMMWYKNSSLLDLEKSRHQIQQTSESFQLSITKVEKPDNGTYSCIAKSSLKTESLDFHLIVKALHEG

SEQ ID NO: 12 - protein, 80 aa, Homo sapiens
TTPGSQASLICAVQNHTREEELLWYREEGRVDLKSGNKINSSSVCVSSISENDNGISFTCRLGRDQSVSVSVVLNVTFPP

SEQ ID NO: 13 - protein, 92 aa, Homo sapiens
LLSGNDFQTVEEGSNVKLVCNVKANPQAQMMWYKNSSLLDLEKSRHQIQQTSESFQLSITKVEKPDNGTYSCIAKSSLKTESLDFHLIVKDK

SEQ ID NO: 14 - DNA, 2295 bp, Homo sapiens
agaccgcgagcgagagcgcccccgagcagcgcccgcgccctccgcgccttctccgccgggacctcgagcgaaagacgcccgcccgccgcccagccctcgcctccctgcccaccgggcccaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccagctccaccgctctgaaggggatctgcagagctcagtcagagggcagaccctgtgaatataactccagaatctaccaaaacggggaaagtttccagcccaactgtaaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgacgaggatagtatcaaggaccccatggaggaccaggacggcctccttggcaaggagctgggattcgatgcctccgaggtggagttgacgagaaacaatgaattgattgcagttggaaaaggcagctcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttgctcattcttgaggagcattaaggtatttcgaaactgccaagggtgctggtgcggatggacactaatgcagccacgattggagaatactttgcttcatagtattggagcacatgttactgcttcattttggagcttgtggagttgatgactttctgttttctgtttgtaaattatttgctaagcatattttctctaggcttttttccttttggggttctacagtcgtaaaagagataataagattagttggacagtttaaagcttttattcgtcctttgacaaaagtaaatgggagggcattccatcccttcctgaagggggacactccatgagtgtctgtgagaggcagctatctgcactctaaactgcaaacagaaatcaggtgttttaagactgaatgttttatttatcaaaatgtagcttttggggagggaggggaaatgtaatactggaataatttgtaaatgattttaattttatattcagtgaaaagattttatttatggaattaaccatttaataaagaaatatttacctaatatctgagtgtatgccattcggtatttttagaggtgctccaaagtcattaggaacaacctagctcacgtactcaattattcaaacaggacttattgggatacagcagtgaattaagctattaaaataagataatgattgcttttataccttcagtagagaaaagtctttgcatataaagtaatgtttaaaaaacatgtattgaacacgacattgtatgaagcacaataaagattctgaagctaaatttgtgatttaagaaaa

SEQ ID NO: 15 - protein, 14 aa, Homo sapiens
VLTVNGKTENYILD

SEQ ID NO: 16 - protein, 7 aa, Homo sapiens
TTPGSQA

SEQ ID NO: 17 - protein, 10 aa, Homo sapiens
SLICAVQNHT

SEQ ID NO: 18 - protein, 10 aa, Homo sapiens
REEELLWYRE

SEQ ID NO: 19 - protein, 10 aa, Homo sapiens
EGRVDLKSGN

SEQ ID NO: 20 - protein, 10 aa, Homo sapiens
KINSSSVCVS

SEQ ID NO: 21 - protein, 10 aa, Homo sapiens
SISENDNGIS

SEQ ID NO: 22 - protein, 10 aa, Homo sapiens
FTCRLGRDQS

SEQ ID NO: 23 - protein, 13 aa, Homo sapiens
VSVSVVLNVTFPP

SEQ ID NO: 24 - protein, 17 aa, Homo sapiens
LLSGNDFQTVEEGSNVK

SEQ ID NO: 25 - protein, 10 aa, Homo sapiens
LVCNVKANPQ

SEQ ID NO: 26 - protein, 10 aa, Homo sapiens
AQMMWYKNSS

SEQ ID NO: 27 - protein, 10 aa, Homo sapiens
LLDLEKSRHQ

SEQ ID NO: 28 - protein, 10 aa, Homo sapiens
IQQTSESFQL

SEQ ID NO: 29 - protein, 10 aa, Homo sapiens
SITKVEKPDN

SEQ ID NO: 30 - protein, 10 aa, Homo sapiens
GTYSCIAKSS

SEQ ID NO: 31 - protein, 10 aa, Homo sapiens
LKTESLDFHL

SEQ ID NO: 32 - protein, 8 aa, Homo sapiens
IVKDKTVG

SEQ ID NO: 33 - protein, 14 aa, Homo sapiens
GLECNFGASSTALK